(19) United States
(12) Patent Application Publication
(10) Pub. No.: US 2025/0320270 A1
(43) Pub. Date: Oct. 16, 2025

(54) POLYPEPTIDE CONSTRUCTS WITH NOVEL BINDING AFFINITY AND USES THEREOF

(71) Applicant: The Board of Trustees of the Leland Stanford Junior University, Sanford, CA (US)

(72) Inventors: Shin-Heng CHIOU, Palo Alto, CA (US); Diane TSENG, Palo Alto, CA (US); Crystal L. MACKALL, Palo Alto, CA (US); Mark M. DAVIS, Palo Alto, CA (US)

(21) Appl. No.: 18/548,765

(22) PCT Filed: Mar. 2, 2022
(86) PCT No.: PCT/US2022/018529

§ 371 Date: Sep. 1, 2023

Related U.S. Application Data
(60) Provisional application No. 63/156,026, filed on Mar. 3, 2021.

(51) Int. Cl. C07K 14/725; A61K 38/00; C12N 5/0783

(52) U.S. Cl. CPC C07K 14/7051; C12N 5/0636; A61K 38/00; C12N 2510/00

(57) ABSTRACT

The present disclosure relates generally to polypeptide constructs, and particularly relate to T-cell receptor (TCR) constructs having binding affinity for a specific cognate antigen. The disclosure also provides compositions and methods useful for producing such constructs as well as methods for the diagnosis, prevention, and/or treatment of conditions associated with cells expressing the cognate antigen recognized by the polypeptide constructs.

### CROSS-REFERENCE TO RELATED APPLICATIONS

This application claims priority to U.S. Provisional Patent Application No. 63/156,026, filed Mar. 3, 2021, the disclosure of which is incorporated by reference herein in its entirety, including any drawings.

### STATEMENT REGARDING FEDERALLY SPONSORED R&D

This invention was made with government support under grant no. U54 CA232568-01 awarded by The National Cancer Institute. The government has certain rights in the invention.

### INCORPORATION OF THE SEQUENCE LISTING

This application contains a Sequence Listing, which is hereby incorporated herein by reference in its entirety. The contents of the electronic sequence listing 2024 Feb. 5 Sequence_Listing_ST26_078430-532N01US.xml; Size: 192,460 bytes; and Date of Creation: Feb. 5, 2024.

### FIELD

The present disclosure relates generally to the field of immunology, and particularly relate to polypeptide constructs having binding affinity for a specific antigen. The disclosure also provides compositions and methods useful for producing such constructs as well as methods for the diagnosis, prevention, and/or treatment of health conditions associated with cells expressing the cognate antigen recognized by the polypeptide constructs.

### BACKGROUND

In recent years, the wide use of immune checkpoint blockade and T cell-based immunotherapies to treat patients with solid tumors requires a deeper understanding of the T cell specificities in cancer. For example, T-cell receptors (TCR) have emerged in recent years as a promising approach for immunotherapy and made headlines in clinical trials conducted by a number of pharmaceutical and biotechnology companies. TCRs have been shown to have therapeutic and diagnostic potential and can be modified similarly to antibody molecules. In particular, the affinity of TCRs for a specific antigen makes them valuable for various therapeutic strategies, including adoptive immunotherapy.

However, despite the widespread use of immunotherapies for treating cancer, general understanding of T cell specificities in cancer is limited. For example, antigen specificity is the key determinant of T cell function, but challenges posed by TCR diversity and human leukocyte antigens (HLA) allele polymorphism have been major obstacles to understanding the full scope of antigens recognized by tumor-infiltrating T cells. In addition, the specificities of the vast majority of tumor-infiltrating T cells remain unknown across all solid tumors despite the availability of advanced technologies for profiling T cell states and repertoires using single-cell sequencing techniques. This is largely due to the absence of tools for analyzing diverse TCR repertoires in the context of highly polymorphic human leukocyte antigens (HLA) alleles. For example, while next-generation sequencing technologies have made the sequencing of large numbers of TCR relatively straightforward and inexpensive, a major problem revolves around how these very large repertoires can be analyzed. This is because there can be hundreds or thousands of possible TCR sequences for the same peptide-MHC specificity.

Accordingly, uncovering the specificities of tumor-infiltrating T cells is important for understanding how T cell-intrinsic factors shape tumor-immune system interactions and impact therapies aimed at harnessing T cell responses against cancer.

### SUMMARY

The present disclosure relates generally to the field of immunology. More particularly, provided herein are novel polypeptide constructs having binding affinity for a specific antigen. The disclosure also provides compositions and methods useful for producing such polypeptide constructs as well as methods for the diagnosis, prevention, and/or treatment of conditions associated with cells expressing the cognate antigen recognized by the polypeptide constructs. In particular, also provided are recombinant cells such as lymphocyte T cells that have been engineered to express a polypeptide construct as disclosed herein and are directed against a cell of interest such as a cancer cell.

In one aspect, provided herein are various constructs including at least one complementary determining region (CDR) having at least 70%, 75%, 80%, 85%, 90%, 95%, 96%, 97%, 98%, 99%, or 100% sequence identity to a sequence selected from the group consisting of SEQ ID NOs: 1-106.

Non-limiting exemplary embodiments of the disclosed constructs can include one or more of the following features. In some embodiments, the at least one CDR has at least 70%, 75%, 80%, 85%, 90%, 95%, 96%, 97%, 98%, 99%, or 100% sequence identity to a sequence selected from the group consisting of SEQ ID NOs: 1-56. In some embodiments, the at least one CDR has at least 70%, 75%, 80%, 85%, 90%, 95%, 96%, 97%, 98%, 99%, or 100% sequence identity to SEQ ID NO: 18. In some embodiments, the at least one CDR has at least 70%, 75%, 80%, 85%, 90%, 95%, 96%, 97%, 98%, 99%, or 100% sequence identity to a sequence selected from the group consisting of SEQ ID NOs: 57-106. In some embodiments, the at least one CDR has at least 70%, 75%, 80%, 85%, 90%, 95%, 96%, 97%, 98%, 99%, or 100% sequence identity to SEQ ID NO: 64. In some embodiments, the construct is single-chain constructs or double-chain constructs. In some embodiments, the construct is selected from the group consisting of: (a) a T cell receptor (TCR); (b) an antibody; and (c) a functional derivative or fragment of (a) or (b). In some embodiments, the construct is a TCR construct including a TCR alpha chain and a TCR beta chain operably linked to each other. In some embodiments, the construct is a TCR construct including in its beta chain a CDR3β having at least 70%, 75%, 80%, 85%, 90%, 95%, 96%, 97%, 98%, 99%, or 100% sequence identity to a sequence selected from SEQ ID NOs: 1-106. In some embodiments, the CDR3β has at least 70%, 75%, 80%, 85%, 90%, 95%, 96%, 97%, 98%, 99%, or 100% sequence identity to a sequence selected from SEQ ID NOs: 1-56. In some embodiments, the CDR3β has at least 70%, 75%, 80%, 85%, 90%, 95%, 96%, 97%, 98%, 99%, or 100% sequence identity to SEQ ID NO: 18. In some embodiments, the CDR3β has at least 70%, 75%, 80%, 85%, 90%, 95%, 96%, 97%, 98%, 99%, or 100% sequence identity to a sequence selected from SEQ ID NOs: 57-106. In some embodiments, the CDR3β has at least 70%, 75%, 80%, 85%, 90%, 95%, 96%, 97%, 98%, 99%, or 100% sequence identity to SEQ ID NO: 64. In some embodiments, the construct further includes in its alpha chain a CDR3a sequence.

In some embodiments, the construct disclosed herein is an antibody construct selected from the group consisting of an antigen-binding fragment (Fab), a single-chain variable fragment (scFv), a nanobody, a single domain antibody (sdAb), a V_{H} domain, a V_{L} domain, a V_{H}H domain, a diabody, or a functional fragment of any thereof.

In another aspect, provided herein are recombinant nucleic acids, wherein the nucleic acids including a nucleic sequence encoding a construct of the disclosure.

Non-limiting exemplary embodiments of the disclosed nucleic acids can include one or more of the following features. In some embodiments, the nucleic acid sequence is operably linked to a heterologous nucleic acid sequence. In some embodiments, the nucleic acid molecule is further configured as an expression cassette or an expression vector. In some embodiments, the vector is a plasmid vector or a viral vector. In some embodiments, the viral vector is derived from a lentivirus, an adeno virus, an adeno-associated virus, a baculovirus, or a retrovirus.

In another aspect, some embodiments of the disclosure relates to engineered cells that include one or more of: (a) a construct of the disclosure and/or (b) a recombinant nucleic acid of the disclosure. Non-limiting exemplary embodiments of the disclosed cells can include one or more of the following features. In some embodiments, the engineered cell is a eukaryotic cell. In some embodiments, the eukaryotic cell is a mammalian cell. In some embodiments, the mammalian cell is a human cell. In some embodiments, the cell is an immune cell. In some embodiments, the immune cell is a B cell, a monocyte, a natural killer (NK) cell, a natural killer T (NKT) cell, a basophil, an eosinophil, a neutrophil, a dendritic cell, a macrophage, a regulatory T cell, a helper T cell (T_{H}), a cytotoxic T cell (T_{CTL}), a memory T cell, a gamma delta (γδ) T cell, another T cell, a hematopoietic stem cell, or a hematopoietic stem cell progenitor.

In some embodiments, the immune cell is a lymphocyte. In some embodiments, the lymphocyte is a T lymphocyte or a T lymphocyte progenitor. In some embodiments, the T lymphocyte is a CD4+ T cell or a CD8+ T cell. In some embodiments, the T lymphocyte is a CD8+ T cytotoxic lymphocyte cell selected from the group consisting of naïve CD8+ T cells, central memory CD8+ T cells, effector memory CD8+ T cells, effector CD8+ T cells, CD8+ stem memory T cells, and bulk CD8+ T cells. In some embodiments, the T lymphocyte is a CD4+ T helper lymphocyte cell selected from the group consisting of naïve CD4+ T cells, central memory CD4+ T cells, effector memory CD4+ T cells, effector CD4+ T cells, CD4+ stem memory T cells, and bulk CD4+ T cells.

In a related aspect, some embodiments of the disclosure relate to cell cultures that include at least one engineered cell of the disclosure and a culture medium.

In another aspect, some embodiments disclosed herein relate to methods for making an engineered cell, wherein the method includes (a) providing a host cell capable of protein expression; and (b) transducing the provided host cell with a recombinant nucleic acid of the disclosure to produce an engineered cell. Accordingly, in a related aspect, also provided herein are engineered cells produced by the methods of the disclosure. In a further related aspect, some embodiments of the disclosure relate to cell cultures that include at least one engineered cell of the disclosure and a culture medium.

In one aspect, provided herein are various pharmaceutical compositions, wherein the pharmaceutical compositions include a pharmaceutically acceptable carrier and one or more of: (a) a construct of the disclosure; (b) a recombinant nucleic acid of the disclosure; and/or (c) an engineered cell of the disclosure.

Non-limiting exemplary embodiments of the disclosed pharmaceutical compositions can include one or more of the following features. In some embodiments, the composition includes a recombinant nucleic acid of the disclosure and a pharmaceutically acceptable carrier. In some embodiments, the recombinant nucleic acid is encapsulated in a viral capsid or a lipid nanoparticle. In some embodiments, the composition includes an engineered cell of the disclosure and a pharmaceutically acceptable carrier.

In another aspect, some embodiments of the disclosure relate to methods for the prevention and/or treatment of a condition in a subject in need thereof, wherein the methods include administering to the subject a composition including one or more of: (a) a construct of the disclosure; (b) a recombinant nucleic acid of the disclosure; (c) an engineered cell of the disclosure; and d) a pharmaceutically composition of the disclosure.

Non-limiting exemplary embodiments of the disclosed methods for preventing and/or treating a condition in a subject in need thereof can include one or more of the following features. In some embodiments, the condition is associated with an immune checkpoint blockade. In some embodiments, the method is for a checkpoint blockade immunotherapy. In some embodiments, the checkpoint blockade immunotherapy is an anti-PD1 checkpoint therapy or an anti-PD1-L1 checkpoint therapy. In some embodiments, the condition is associated with a lung cancer is selected from the group consisting of adenocarcinoma, squamous cell carcinoma, small cell carcinoma, non-small cell carcinoma, adenosquamous carcinoma, small cell lung cancer, large cell carcinoma, neuroendocrine cancers of the lung, non-small cell lung cancer (NSCLC), undifferentiated non-small cell carcinoma, non-small cell carcinoma not otherwise specified, pulmonary squamous cell carcinoma, broncho-alveolar carcinoma, sarcomatoid carcinoma, pleomorphic carcinoma, carcinosarcoma, pulmonary blastoma, metastatic carcinoma of unknown primary, primary pulmonary lymphoepithelioma-like carcinoma, and benign neoplasms of the lung. In some embodiments, the lung cancer is a NSCLC selected from the group consisting of squamous cell carcinoma, adenocarcinoma, large cell carcinoma, carcinoid tumor, pleomorphic, salivary gland cancer, adenosquamous, sarcomatoid, and unclassified carcinomas. In some embodiments, the NSCLC includes stage I NSCLC or stage II NSCLC. In some embodiments, the cancer is a non-metastatic cancer, a metastatic cancer, a multiply drug resistant cancer, or a recurrent cancer. In some embodiments, the administered composition inhibits tumor growth or metastasis of the cancer in the subject.

In some embodiments, provided herein are methods for preventing and/or treating a condition in a subject in need thereof, wherein the condition is a malignancy associated with a viral infection. In some embodiments, the condition is a malignancy associated with an infection by Epstein-Barr virus (EBV). In some embodiments, the malignancy is associated with an EBV infection and is selected from the group consisting of Hodgkin lymphoma, Burkitt lymphoma, diffuse large B cell lymphoma, nasopharyngeal carcinoma, gastric carcinoma, post-transplant lymphoproliferative disease, B lymphoproliferative disease, T/NK lymphoproliferative disease, T/NK lymphomas/leukemias, leiomyosarcomas, and lymphoepithelioma-like carcinomas.

In some embodiments of the methods for preventing and/or treating a condition in a subject in need thereof, wherein the subject is a mammal. In some embodiments, the mammal is a human. In some embodiments, the composition is administered to the subject individually as a first therapy (monotherapy) or in combination with at least one additional therapies. In some embodiments, the at least one additional therapies is selected from the group consisting of chemotherapy, radiotherapy, immunotherapy, hormonal therapy, toxin therapy, targeted therapy, or surgery. In some embodiments, the at least one additional therapies is selected from the group consisting of an anti-CTLA4 antibody, an anti-PD-1 antibody, an anti-PD-L1 antibody, an anti-CD20 antibody, an anti-CD40 antibody, an anti-DR5 antibody, an anti-CDId antibody, an anti-TIM3 antibody, an anti-SLAMF7 antibody, an anti-KIR receptor antibody, an anti-OX40 antibody, an anti-HER2 antibody, an anti-ErbB-2 antibody, an anti-EGFR antibody, cetuximab, rituximab, trastuzumab, pembrolizumab, radiotherapy, single dose radiation, fractionated radiation, focal radiation, whole organ radiation, IL-12, IFNα, GM-CSF, a chimeric antigen receptor, adoptively transferred T cells, an anti-cancer vaccine, and an oncolytic virus. In some embodiments, the first therapy and the at least one additional therapies are administered concomitantly. In some embodiments, the first therapy is administered at the same time as the at least one additional therapies. In some embodiments, the first therapy and the at least one additional therapies are administered sequentially. In some embodiments, the first therapy is administered before the at least one additional therapies. In some embodiments, the first therapy is administered after the at least one additional therapies. In some embodiments, the first therapy is administered before and/or after the at least one additional therapies. In some embodiments, the first therapy and the at least one additional therapies are administered in rotation. In some embodiments, the first therapy and the at least one additional therapies are administered together in a single formulation.

In another aspect, some embodiments of the disclosure relate to kits for the practice of the methods disclosed herein. Some embodiments relate to kits for methods of the diagnosis, prevention, and/or treatment a condition in a subject in need thereof, wherein the kits include one or more of: a construct of the disclosure; a recombinant nucleic acid of the disclosure; an engineered cell of the disclosure; and a pharmaceutical composition of the disclosure.

In another aspect, provided herein is the use of one or more of: a construct of the disclosure; a recombinant nucleic acid of the disclosure; an engineered cell of the disclosure; and a pharmaceutical composition, for the prevention and/or treatment of a condition. In some embodiments, the condition is a proliferative disorder. In some embodiments, the proliferative disorder is a cancer. In some embodiments, the condition is a malignancy associated with an infection. In some embodiments, the infection is a bacterial infection or viral infection.

In another aspect, provided herein is the use of one or more of: a construct of the disclosure, a recombinant nucleic acid of the disclosure, an engineered cell of the disclosure, or a pharmaceutical composition of the disclosure, in the manufacture of a medicament for the treatment of a health condition. In some embodiments, the condition is a proliferative disorder. In some embodiments, the proliferative disorder is a cancer. In some embodiments, the condition is a malignancy associated with an infection. In some embodiments, the infection is a bacterial infection or viral infection.

In yet another aspects, provided herein are various methods for obtaining a construct as disclosed herein, the methods include (a) identifying a plurality of T cell receptors (TCRs) associated with a health condition; (b) determining a sequence of a CDR3β present in each of the identified TCRs; and (c) making a construct including a CDR3β sequence determined in (b), wherein the construct is capable of binding to the one or more cognate antigens. In some embodiments, the condition is a proliferative disease. In some embodiments, the method further includes identifying one or more antigens commonly recognized by the CDR3β sequences.

The foregoing summary is illustrative only and is not intended to be in any way limiting. In addition to the illustrative embodiments and features described herein, further aspects, embodiments, objects and features of the disclosure will become fully apparent from the drawings and the detailed description and the claims.

### BRIEF DESCRIPTION OF THE DRAWINGS

FIGS. 1A-1C schematically summarize the results of experiments performed to establish specificity groups with TCR CDR3β sequences from lung cancer patients. FIG. 1A: Schematic of the four steps involved in TCR specificity inference with the GLIPH2 algorithm (Grouping of Lymphocyte Interactions by Paratope Hotspots). Step 1 involves acquisition of T cell receptor CDR3β sequences. In this step, 778,938 CDR3β sequences from the MDACC cohort were used as input for GLIPH2 analysis. Step 2 involves discovery of short sequence motifs within CDR3β sequences from multiple patients. These shared motifs are predicted to be involved in the direct engagement with antigenic peptides loaded on HLA molecules. In this step, 66,094 specificity groups with multiple criteria were established (see also, FIG. 3A). Step 3 involves establishment of 4,226 clonally expanded specificity groups. Multiple cutoffs are used for the inference of a given specificity group, including a) the enrichment of Vβ genes, b) minimum numbers of distinct CDR3β sequences=3, c) minimum numbers of patients=3, d) enrichment of clonally expanded CDR3β clonotypes, and e) enrichment of HLA alleles. Step 4 involves establishment of 435 clonally expanded, tumor-enriched specificity groups. FIG. 1B: Relevance of tumor-enriched specificity groups in lung cancer. The most expanded CDR3β sequences from tumors belonged to the 435 tumor-enriched specificity groups, whereas those from lung tissues of healthy donors and COPD patients did not. The trend was validated with tumors of a second NSCLC cohort (the TRACERx consortium, n=202, validation). ***, p<0.001; *, p<0.05 by paired t test. NS, not significantly different. FIG. 1C: Network analysis of 396 specificity groups annotated with CDR3β sequences from HLA tetramers with influenza virus (Flu, red), Epstein-Barr virus (EBV, green), and cytomegalovirus (CMV, blue) antigens. Each dot is a specificity group, edges indicate the presence of identical CDR3β sequence(s) across two specificity groups.

FIGS. 2A-2C schematically summarize the results of experiments performed to illustrate virus-specific CD8+ T cell clones expanded in patients responding to anti-PD1 treatment. FIG. 2A: Comparisons of pre- and post-treatment CDR3β clonal frequencies in the peripheral blood of patient M1 (left) and M2 (right). CDR3β clones inferred to recognize viral antigens are highlighted. FIG. 2B: Specificity groups containing expanded CDR3β clones post-treatment (column 5, CDR3β sequence) from patients MI or M2 (column 6, Patient ID) that are annotated with viral tetramer CDR3β sequences (column 2-4, antigen and HLA alleles of the tetramers). Enrichment of the A*02:01 or B*35:01 allele is shown (last 2 columns, p values from the hypergeometric tests are shown). CDR3α/β sequences of the two EBV-related expanded clones from patient M2 are shown at the bottom. FIG. 2C: TCR27-Jurkat cell line (CDR3β: CASSTGDSNQPQHF; SEQ ID NO: 64, top panels) and TCR28-Jurkat cell line (CDR3β: CASSARTGELFF; SEQ ID NO: 18, bottom panels) were created and tested for their reactivities to the predicted EBV antigens in the context of B*35 as shown in FIG. 2B. TCR27- and TCR28-Jurkat cells were co-cultured with T2-B*35 cells pulsed with indicated peptides (above each plot). Level of activation was quantified with CD69 expression. In these experiments, the control peptide had the following sequence: LPFDFTPGY (SEQ ID NO: 107).

FIG. 3A: Specificity inference pipeline, which schematically summarizes the data availability for the 178 HLA-typed NSCLC patients from the MD Anderson Cancer Center (MDACC). FIG. 3B: Low percentages of TCR clonotypes from adjacent lung tissues are grouped into tumor-enriched specificity groups. The percentages of the top 20 most expanded CDR3β clonotypes from the adjacent lung tissues of patients belonging to the MDACC NSCLC cohort (n=178 samples, left) and the TRACERx cohort (n=63 samples, right) were quantified for those that belonged to the 435 tumor-enriched specificity groups as in FIG. 1B (% grouped, log10-converted). The same analysis was performed on the remainder of the CDR3β clonotypes (Non-exp, non-expanded). ND, no statistically significant difference was found. FIG. 3C: The 71 clonally expanded specificity groups annotated and colored with 10 indicated tetramers are shown in the network. FIG. 3D schematically summarizes the in silico validation of TCR specificity groups using HLA tetramer sequences. Left to right, network analysis of 71 clonally expanded specificity groups colored as in FIG. 1C is shown; the two large Flu-related communities (red) are circled and the CDR3β members of the specificity groups are highlighted with the previously reported short motifs “RS” and “GxY” highlighted in red font; heatmap showing distinct CDR3β members (columns) of the Flu-related (with the “RS” motif) specificity groups (rows) and the levels of shared CDR3β members between specificity groups within the circled community; table showing an example of the “SIRSS % E” specificity group containing the short “RS” motif (bold) that is annotated with 5 Flu-specific tetramer sequences (bottom). The counts of distinct CDR3β members from tumor and the Vβ gene usage are shown (top). FIG. 3E: 394 specificity groups annotated with indicated tetramers (key) were organized into distinct communities through shared CDR3β sequence(s) as in FIG. 1C. Thickness of edge represents numbers of shared CDR3β sequence(s) between any two connected nodes. FIG. 3F: Community plot as in FIG. 3E. Color of edge represents shared CDR3β sequence(s) between specificity groups with identical (red) or distinct (blue) specificities defined by tetramer-derived sequences (labeled with distinct colors, E). 588 of all (n=634) connections (edges) are labeled in red (92.74%). FIG. 3G: 71 clonally expanded specificity groups as in FIG. 3C. Color of edge represents shared CDR3β sequence(s) between specificity groups with identical (red) or distinct (blue) specificities defined by tetramer-derived sequences (FIG. 3C). 92 of all (n=92) connections (edges) are labeled in red (100%).

FIGS. 4A-4B schematically summarize the results of CDR3β sequences inferred to recognize CMV, Flu, and EBV do not differ in their distribution between tumor and uninvolved lung. (A) Volcano plots showing the relative distributions of CDR3β sequences with inferred specificities to CMV (blue), Flu (red), or EBV (green) across the tumor (T) and uninvolved lung (N) by comparing multiple patients with Poisson test. The y-axis shows the negative log10 converted p values of the Poisson test and the x-axis shows the log2 converted fold-difference between tumor and uninvolved (adjacent) lung (T/N). (B) Total frequencies of clonotypes in tumor (right) or uninvolved lung (left) that are inferred to recognize antigens from EBV, CMV, or Flu by GLIPH2. Each dot is a patient and total frequencies are shown as log10 converted values (first and the third quartiles show 25th & 75th percentiles, respectively).

FIG. 5 is a schematic of the combined single-cell TCR-Seq and single-cell RNA-Seq (scRNA-seq) procedures. CD45+CD3+ T cells were sorted from single-cell suspensions of lung tumor samples from patients with NSCLC at Stanford. Single-cell TCR-Seq was performed using nested multiplexed PCR as previously described (Han et al., 2014. Nat. Biotechnol. 32, 684). Single-cell RNseq was performed according to previous methods (Picelli et al., 2014. Nat. Protoc. 9, 171) with modifications as details in the methods. TCR repertoires were integrated from the single-cell TCR-Seq pipeline and from the scRNA-seq data with reconstruction using the TraCeR algorithm (Stubbington et al., 2016. Nat. Methods 13, 329) for GLIPH2 analysis.

FIG. 6A shows CT scan images of pre- and post-treatment from NSCLC patient MI (top panels) and M2 (bottom panels) treated with anti-PD1 therapy. Tumors are highlighted with red arrowheads. FIG. 6B: T2 (174×CEM.T2) cells were transduced with lentiviral vector encoding the full-length coding sequence of WT human HLA-B*35:01. Cells were selected with puromycin and the surface B*35 expression was quantified by FACS with or without the control peptide “LPFDFTPGY” (SEQ ID NO: 107) reported previously (Takamiya et al., 1994. Int Immunol. Vol. 6, 255). FIG. 6C is a volcano plot showing the comparison of the 66,094 shared specificity groups between tumor (T) and the adjacent lung (N) by Poisson test. The y-axis represents the negative log10 converted p values of the Poisson tests and the x-axis represents the log2 converted fold difference between tumor and the adjacent lung (T/N). Dot size represents levels of clonal expansion. Specificity groups annotated with pathogen-related tetramer CDR3β sequences as in FIG. 2B (n=11) are highlighted according to the respective CDR3β sequences of the expanded clones (5th column, FIG. 2B).

### DETAILED DESCRIPTION OF THE DISCLOSURE

The present disclosure generally relates to, inter alia, compositions and methods for the diagnosis, prevention, and/or treatment of health conditions. More particularly, provided herein are novel polypeptide constructs having binding affinity for a specific cognate antigen. The disclosure also provides compositions and methods useful for producing such polypeptide constructs as well as methods for the diagnosis, prevention, and/or treatment of conditions associated with cells expressing the cognate antigen recognized by the polypeptide constructs. In particular, also provided are recombinant cells such as lymphocyte T cells that have been engineered to express a polypeptide construct as disclosed herein and are directed against a cell of interest such as a cancer cell. As will be discussed more thoroughly below, the present disclosure describes an approach that combines bioinformatics and antigen screening to identify novel shared tumor antigens in lung cancer. In some embodiments, the disclosed approach implements an improved version of the algorithm GLIPH (Grouping of Lymphocyte Interactions with Paratope Hotspots), GLIPH2 ([23] and [24]), to infer the T cell specificities for shared antigens at a global level. Using TCR repertoires from 178 HLA-typed lung cancer patients, GLIPH2 identified over 400 specificity groups inferred to recognize shared tumor antigens in defined HLA contexts. Subsequent analyses were then performed on those with inferred HLA-B*35 restrictions, which informed the prioritization of two particular specificity groups, TCR27 and TCR28. As described in greater detail below, additional analyses revealed that the specificity group TCR27 carries the following motifs for antigen identification: “STGD% NQP”, “% TGDSNQP”, “ST % DSNQP”, “STG % SNQP”, and “S % GDSNQP” where “%” denotes the amino acid that varied (Gee et al., 2018). Non-limiting exemplary CDR3β sequences of the TCR27 specifity group include, for example, those provided in the Sequence Listing as SEQ ID NO: 57-106. The specificity group TCR28 carries the following motifs: “SARTG %”, “S % RTGE”, “SAR % GE”, “SA % TGE”, and “SART % E”. Non-limiting exemplary CDR3β sequences of the TCR28 specifity group include, for example, those provided in the Sequence Listing as SEQ ID NO: 1-56.

As discussed above, the wide use of immune checkpoint blockade and T cell-based immunotherapies to treat patients with solid tumors requires a deeper understanding of the T cell specificities in cancer. However, the specificities of the vast majority of tumor-infiltrating T cells remain unknown across all solid tumors despite the availability of advanced technologies for profiling T cell states and repertoires using single-cell sequencing techniques. In recent years, the handful specificities of tumor-infiltrating T cells that have been previously described include T cells recognizing mutated antigens, non-mutated (shared) antigens, and viral antigens. In the era of immune checkpoint blockade, there has been a recent focus on mutated antigens (e.g., neoantigens). As neoantigens represent a type of “altered self” antigen, T cells recognizing this class of antigens have been shown to exhibit an activated phenotype and respond vigorously in tumors. Non-mutated tumor antigens include differentiation antigens (e.g. melanoma-associated antigens) that are expressed in normal tissue counterparts, or self-antigens where expression is restricted to immune-privileged sites, germline tissue, or embryos. There have been numerous examples targeting these types of tumor antigen with adoptive T cell therapies. In addition, T cells with specificities for viruses (such as HPV, EBV, and Merkel cell polyomavirus) have also been a focus of investigation for virus-associated cancers.

In contrast, the role of other types of T cell specificities in solid tumors remains elusive. For example, numerous reports have described the existence of virus-specific T cells in tumors, such as T cells specific for influenza virus (Flu) or cytomegalovirus (CMV) in lung cancer. Without direct evidence of such viruses playing a role in the oncogenesis of lung cancer or other solid tumors, they have largely been presumed to be irrelevant to the tumor immune response and are often referred as “bystander cells”. As described in greater detail below, experimental data described herein have identified a class of specific CD8+ T cells and their cross-reactive antigens from cancer cells and pathogens. This finding is consistent with the hypothesis that maintaining a broad T cell repertoire to defend against viruses and other pathogens may rely on cross-reactivity. T cells specific to self-antigens have been detected in the peripheral blood of healthy individuals, pruned but not clonally deleted in the thymus, potentially to avoid immunologic “blind spots” to viruses and other pathogens. Because cancer cells histologically resemble their tissue of origin and can express self-antigens, experiments have been designed to investigate the possibility that some tumor-infiltrating T cells are indeed specific to ubiquitously expressed, non-mutated self-antigens. Comprehensively profiling and deep characterization of T cell specificities within the tumor microenvironment provides a fundamental understanding of the T cell response beyond phenotypic characterization and sheds important insight on how the immune system recognizes tumors, normal tissues, and pathogens.

The vast majority of tumor-infiltrating T cells remain unknown is largely due to the absence of tools for analyzing diverse TCR repertoires in the context of highly polymorphic human leukocyte antigens (HLA) alleles. For example, while next-generation sequencing technologies have made the sequencing of large numbers of TCR relatively straightforward and inexpensive, a major problem revolves around how these very large repertoires can be analyzed.

This is because there can be hundreds or thousands of possible TCR sequences for the same peptide-MHC specificity. GLIPH algorithms (Glanville et al., 2018), and more recently an improved version (GLIPH2; Huang et al, 2020) have been previously developed to systemically profile antigen specificities of T cells and to allow inferences of T cell specificity solely based on the CDR3β sequences. These algorithms analyze large numbers of sequences quickly and parse them into TCR specificity groups (a.k.a. specificity groups) that can predict the likely MHC allele restriction. As described in greater detail below, GLIPH2 was used to analyze 778,938 distinct TCRβ CDR3 sequences (referred to as CDR3β sequences) from 178 HLA-typed, non-small cell lung cancer (NSCLC) patients with surgically resectable disease. A total of 4,300 high-confidence specificity groups were initially derived. Of those, 449 were found enriched in tumor compared to uninvolved lung tissue. It was also found that up to 35% of all tumor-infiltrating T cell repertoires within a patient were inferred to have shared antigen specificities. Subsequently, select specificity groups were validated by identifying novel clonotypes predicted to recognize known viral antigens in given HLA contexts and experimentally confirmed these predictions. Next, two specificity groups were prioritized that were preferentially enriched in tumor and inferred to recognize antigen in the context of HLA-B*35. Phenotypically, these cross-reactive CD8+ T cells adopted an effector cell state, expressing some genes found on activated NK cells and did not express exhaustion markers PD-1 or CD39. In summary, the experimental data described herein offer direct evidence that the T cells infiltrating tumors may cross-react to recognize tumor antigens and pathogen-derived antigens.

As described in greater detail below, the experimental data disclosed herein establishes a novel approach for discovering shared tumor antigens and the T cells that recognize them. In particular, some experimental data presented herein illustrates EBV-specific CDR3β sequences that were clonally expanded in patients who had clinical responses to anti-PD-1 treatment. This suggests that pathogen cross-reactivity may be an important feature in the interaction between neoplasia and T cell immunity. Overall, the data disclosed herein illustrates a generalizable approach to comprehensively analyze shared T cell specificities in human cancer and identify specific antigens using a yeast display library. This data not only serves as a resource for further T cell studies in lung cancer but can also explain why some apparently “random” virus-specific T cells might congregate in the tumor microenvironment and suggests a way in which this might contribute to neoplasia

A non-limiting workflow for the approach for discovering novel shared tumor antigens in a target cancer, e.g., lung cancer, generally begins with comprehensive profiling of the T cell specificity landscape in human lung cancer. The bioinformatics tool GLIPH2 was used to profile 778,938 CDR3β sequences from 178 patients and establish 449 tumor-enriched specificity groups. Two such TCRs with inferred specificity in the context of HLA-B*35 was identified. The platform for T cell antigen identification as disclosed herein brings together two technologies. First, the GLIPH2 algorithm performs unbiased inferences of global T cell specificities with accurate predictions of HLA restriction. More information regarding the GLIPH2 algorithm can be found in Huang et al., Nat Biotechnol, 2020, the content of which is expressed incorporated by reference. The inferences of shared specificity and HLA context are used to prioritize disease-relevant TCR candidates for downstream antigen discovery. Second, the rich diversity of yeast display libraries greatly facilitates antigen identification and allows for discovery of cross-reactive antigens. Unlike other MHC/peptide libraries built in mammalian cells, the yeast display libraries used the experiments described below incorporate more than 10⁸ randomly permutated peptide sequences. Previously, the uncertainty of HLA restriction limited the success of antigen identification using the yeast display libraries. The studies described herein overcome this limitation by using GLIPH2 algorithm to infer the correct HLA context of the candidate TCR prior to screening the yeast library for its antigens.

As discussed above, uncovering the specificities of tumor-infiltrating T cells is important for understanding how T cell-intrinsic factors shape tumor-immune system interactions and impact therapies aimed at harnessing T cell responses against cancer. Complementing the current understanding of T cell exhaustion as a mechanism of tumor immune evasion, the studies described herein demonstrate that T cell specificities for self-antigens also play a role. Without being bound to any particular theory, it is believed that T cell specificity for self-antigens partly explain why previous studies observed low reactivities of tumor-infiltrating T cells to autologous tumor.

In addition, the concept that immunologic exposure to environmental pathogens may influence the immune response to tumors has been previously theorized, although its mechanism is poorly understood. As early as the late 19th century, William Coley pioneered a mixed bacterial vaccine termed Coley's toxin for the treatment of cancer patients with some successes. In the modern era, Bacillus Calmette-Guerin (BCG) is routinely used as an immunotherapy for early-stage bladder cancer. Recently the gut microbiome has been shown to be a key determinant of immunotherapy responses in cancer. In pancreatic cancer, a unique microbiome has been observed in patients with longest survival after surgery. While the mechanism of action of these various examples could involve cell types of the innate immune system, cross-reactive T cells recognizing both tumor and pathogens might be playing an essential role. Furthermore, as the lungs are exposed to respiratory pathogens, it is contemplated that the cross talk between these antigens and tumor antigens is particularly important for understanding the adaptive immune responses to lung cancer.

Experimental results described herein have demonstrated that the categorization of T cell specificities in tumors as tumor-specific or as pathogen-specific bystanders does not fully capture all possibilities for T cell antigen recognition. As described in greater detail below, T cells in tumors can also be cross-reactive to both tumor antigens and pathogen-derived antigens and therefore offers a more nuanced understanding of T cell specificity in tumors. The disclosed approach for finding this particular class of TCRs also demonstrates a novel methodology for discovering additional tumor antigens. This is because a deeper understanding of how cross-reactive T cells recognize tumor antigens and pathogen-derived antigens can inform advancements in cellular therapies, checkpoint therapies, and vaccination strategies against cancer. The experimental data disclosed herein indicates that an individual's encounters with environmental pathogens may shape the adaptive immune response against cancer, a concept that can be harnessed for improving immunotherapies for patients.

### Definitions

Unless otherwise defined, all terms of art, notations and other scientific terms or terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

The singular form “a”, “an”, and “the” include plural references unless the context clearly dictates otherwise. For example, the term “a cell” includes one or more cells, including mixtures thereof. “A and/or B” is used herein to include all of the following alternatives: “A”, “B”, “A or B”, and “A and B”.

The term “about”, as used herein, has its ordinary meaning of approximately. If the degree of approximation is not otherwise clear from the context, “about” means either within plus or minus 10% of the provided value, or rounded to the nearest significant figure, in all cases inclusive of the provided value. Where ranges are provided, they are inclusive of the boundary values.

The terms “administration” and “administering”, as used herein, refer to the delivery of a bioactive composition or formulation by an administration route including, but not limited to, oral, intravenous, intra-arterial, intramuscular, intraperitoneal, subcutaneous, intramuscular, and topical administration, or combinations thereof. The term includes, but is not limited to, administering by a medical professional and self-administering.

The terms “cell”, “cell culture”, “cell line” refer not only to the particular subject cell, cell culture, or cell line but also to the progeny or potential progeny of such a cell, cell culture, or cell line, without regard to the number of transfers or passages in culture. It should be understood that not all progeny are exactly identical to the parental cell. This is because certain modifications may occur in succeeding generations due to either mutation (e.g., deliberate or inadvertent mutations) or environmental influences (e.g., methylation or other epigenetic modifications), such that progeny may not, in fact, be identical to the parent cell, but are still included within the scope of the term as used herein, so long as the progeny retain the same functionality as that of the originally cell, cell culture, or cell line.

The term “effective”, “therapeutically effective”, or “pharmaceutically effective” amount or number of a subject construct, nucleic acid, cell, or composition of the disclosure generally refer to an amount or number sufficient for a construct, nucleic acid, cell, or composition to accomplish a stated purpose relative to the absence of the composition (e.g., achieve the effect for which it is administered, prevent or treat a disease, inhibit a microbial infection, or reduce one or more symptoms of a health condition). An example of an effective amount or number is an amount or number sufficient to contribute to the treatment, prevention, or reduction of a symptom or symptoms of a disease, which could also be referred to as a therapeutically effective amount. A “reduction” of a symptom(s) generally means decreasing of the severity or frequency of the symptom(s), or elimination of the symptom(s). The exact amount or number of a construct, nucleic acid, cell, or composition will depend on the purpose of the treatment, and can be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

The term “operably linked”, as used herein, denotes a physical or functional linkage between two or more elements, e.g., polypeptide sequences or polynucleotide sequences, which permits them to operate in their intended fashion. For example, the term “operably linked” when used in context of the orthogonal DNA target sequences described herein or the promoter sequence in a nucleic acid construct, or in an engineered response element means that the orthogonal DNA target sequences and the promoters are in-frame and in proper spatial and distance away from a polynucleotide of interest coding for a protein or an RNA to permit the effects of the respective binding by transcription factors or RNA polymerase on transcription. It should be understood that, operably linked elements may be contiguous or non-contiguous.

In the context of polypeptide constructs, “operably linked” refers to a physical linkage (e.g., directly or indirectly linked) between amino acid sequences (e.g., different segments, portions, or domains) to provide for a described activity of the constructs. In the present disclosure, region, or domains of the constructs of the disclosure may be operably linked to retain proper folding, processing, targeting, expression, binding, and other functional properties of the constructs in the cell. Unless stated otherwise, the segments, portions, and domains of the constructs of the disclosure are operably linked to each other. Operably linked segments, portions, and domains of the constructs disclosed herein may be contiguous or non-contiguous (e.g., linked to one another through a linker).

The term “percent identity,” as used herein in the context of two or more nucleic acids or proteins, refers to two or more sequences or subsequences that are the same or have a specified percentage of nucleotides or amino acids that are the same (e.g., about 60% sequence identity, 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection. See e.g., the NCBI web site at ncbi.nlm.nih.gov/BLAST. Such sequences are then said to be “substantially identical.” This definition also refers to, or may be applied to, the complement of a sequence. This definition also includes sequences that have deletions and/or additions, as well as those that have substitutions. Sequence identity can be calculated using published techniques and widely available computer programs, such as the GCS program package (Devereux et al, Nucleic Acids Res. 12:387, 1984), BLASTP, BLASTN, FASTA (Atschul et al., J Mol Biol 215:403, 1990). Sequence identity can be measured using sequence analysis software such as the Sequence Analysis Software Package of the Genetics Computer Group at the University of Wisconsin Biotechnology Center (1710 University Avenue, Madison, Wis. 53705), with the default parameters thereof.

The term “pharmaceutically acceptable excipient” as used herein refers to any suitable substance that provides a pharmaceutically acceptable carrier, additive or diluent for administration of a compound(s) of interest to a subject. As such, “pharmaceutically acceptable excipient” can encompass substances referred to as pharmaceutically acceptable diluents, pharmaceutically acceptable additives, and pharmaceutically acceptable carriers. As used herein, the term “pharmaceutically acceptable carrier” includes, but is not limited to, saline, solvents, dispersion media, coatings, antibacterial and antifungal agents, isotonic and absorption delaying agents, and the like, compatible with pharmaceutical administration. Supplementary active compounds (e.g., antibiotics and additional therapeutic agents) can also be incorporated into the compositions.

As used herein, a “subject” or an “individual” includes animals, such as human (e.g., human individuals) and non-human animals. In some embodiments, a “subject” or “individual” is a patient under the care of a physician. Thus, the subject can be a human patient or an individual who has, is at risk of having, or is suspected of having a disease of interest (e.g., cancer) and/or one or more symptoms of the disease. The subject can also be an individual who is diagnosed with a risk of the condition of interest at the time of diagnosis or later. The term “non-human animals” includes all vertebrates, e.g., mammals, e.g., rodents, e.g., mice, non-human primates, and other mammals, such as e.g., sheep, dogs, cows, chickens, and non-mammals, such as amphibians, reptiles, etc.

The term “vector” is used herein to refer to a nucleic acid molecule or sequence capable of transferring or transporting another nucleic acid molecule. The transferred nucleic acid molecule is generally linked to, e.g., inserted into, the vector nucleic acid molecule. Generally, a vector is capable of replication when associated with the proper control elements. The term “vector” includes cloning vectors and expression vectors, as well as viral vectors and integrating vectors. An “expression vector” is a vector that includes a regulatory region, thereby capable of expressing DNA sequences and fragments in vitro and/or in vivo. A vector may include sequences that direct autonomous replication in a cell, or may include sequences sufficient to allow integration into host cell DNA. Useful vectors include, for example, plasmids (e.g., DNA plasmids or RNA plasmids), transposons, cosmids, bacterial artificial chromosomes, and viral vectors. Useful viral vectors include, e.g., replication defective retroviruses and lentiviruses. In some embodiments, a vector is a gene delivery vector. In some embodiments, a vector is used as a gene delivery vehicle to transfer a gene into a cell.

It is understood that aspects and embodiments of the disclosure described herein include “comprising”, “consisting”, and “consisting essentially of” aspects and embodiments.

As used herein, “comprising” is synonymous with “including”, “containing”, or “characterized by”, and is inclusive or open-ended and does not exclude additional, unrecited elements or method steps. As used herein, “consisting of” excludes any elements, steps, or ingredients not specified in the claimed composition or method. As used herein, “consisting essentially of” does not exclude materials or steps that do not materially affect the basic and novel characteristics of the claimed composition or method. Any recitation herein of the term “comprising”, particularly in a description of components of a composition or in a description of steps of a method, is understood to encompass those compositions and methods consisting essentially of and consisting of the recited components or steps.

Where a range of values is provided, it is understood by one having ordinary skill in the art that all ranges disclosed herein encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to”, “at least”, “greater than”, “less than”, and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth. Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Headings, e.g., (a), (b), (i) etc., are presented merely for case of reading the specification and claims. The use of headings in the specification or claims does not require the steps or elements be performed in alphabetical or numerical order or the order in which they are presented.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

### T Cell Receptors

A TCR is a heterodimeric cell surface protein of the immunoglobulin super-family, which is associated with invariant proteins of the CD3 complex involved in mediating signal transduction. TCRs and antibodies are molecules that have evolved to recognize different classes of antigens (ligands). TCRs are antigen-specific molecules that are responsible for recognizing antigenic peptides presented in the context of a product of the major histocompatibility complex (MHC) on the surface of antigen presenting cells (APCs) or any nucleated cell (e.g., all human cells in the body, except red blood cells). In contrast, antibodies generally recognize soluble or cell-surface antigens, and do not require presentation of the antigen by an MHC. This system endows T cells, via their TCRs, with the potential ability to recognize the entire array of intracellular antigens expressed by a cell (including viral and bacterial proteins) that are processed intracellularly into short peptides, bound to an intracellular MHC molecule, and delivered to the surface as a peptide-MHC complex (pepMHC). This system allows virtually any foreign protein (e.g., mutated cancer antigen or virus protein) or aberrantly expressed protein to serve a target for T cells.

Generally, TCRs exist in αβ and γδ forms, which are structurally similar but have quite distinct anatomical locations and probably functions. The extracellular portion of native heterodimeric αβ TCR generally consists of two polypeptides, an α chain and a β chain, each of which has a membrane-proximal constant domain, and a membrane-distal variable domain. Each of the constant and variable domains includes an intra-chain disulfide bond. The variable domains contain the highly polymorphic loops analogous to the complementarity determining regions (CDRs) of antibodies, embedded in a framework sequence, one being the hyper-variable region named CDR3. There are several types of alpha chain variable (Vα) regions and several types of beta chain variable (Vβ) regions distinguished by their framework, CDR1 and CDR2 sequences, and by a partly defined CDR3 sequence. The use of TCR gene therapy overcomes a number of current hurdles. For example, it allows equipping patients' own T cells with desired specificities and generation of sufficient numbers of T cells in a short period of time, avoiding their exhaustion. In addition, the TCR can be transduced into central memory T cells or T cells with stem cell characteristics, which may ensure better persistence and function upon transfer. Furthermore, TCR-engineered T cells can be infused into cancer patients rendered lymphopenic by chemotherapy or irradiation, allowing efficient engraftment but inhibiting immune suppression.

### Compositions of the Disclosure

As described in greater detail below, one aspect of the present disclosure relates to novel polypeptide constructs having binding affinity for a specific cognate antigen. Also provided are recombinant nucleic acids encoding such polypeptide constructs, as well as recombinant cells that have been engineered to express a polypeptide construct as disclosed herein and are directed against a cell of interest such as a cancer cell.

### A. Constructs of the Disclosure

In one aspect, provided herein are various constructs including at least one complementary determining region (CDR) having at least 70% sequence identity to a sequence selected from the group consisting of SEQ ID NOs: 1-106.

Non-limiting exemplary embodiments of the disclosed constructs can include one or more of the following features. In some embodiments, the constructs include at least one, at least two, or at least three CDR having at least 70% sequence identity to a sequence selected from the group consisting of SEQ ID NOs: 1-106. In some embodiments, the constructs include at least one, at least two, or at least three CDR having at least 70% sequence identity to the sequence of SEQ ID NO: 6. In some embodiments, the constructs include at least one CDR having at least 70%, for example at least 75%, at least 80%, at least 85%, at least 90%, at least 95%, at least 96%, at least 97%, at least 98%, at least 99%, or at least 100% sequence identity to a sequence selected from the group consisting of SEQ ID NOs: 1-106. In some embodiments, the constructs include at least one CDR having at least 70%, for example at least 75%, at least 80%, at least 85%, at least 90%, at least 95%, at least 96%, at least 97%, at least 98%, at least 99%, or at least 100% sequence identity to the sequence of SEQ ID NO: 6.

The CDR sequence of the constructs disclosed herein may be modified, e.g., mutated. Non-limiting examples of modifications of the CDR sequence include a substitution, a deletion, an addition, or an insertion of no more than five, no more than four, no more than three, no more than two, or no more than one amino acid residue. In some embodiments, the at least one CDR of the constructs disclosed herein includes a sequence having 100% identity to a sequence selected from the group consisting of SEQ ID NOs: 1-106, wherein at least 1, at least 2, at least 3, at least 4, at least 5 amino acid residues in the sequence is substituted by a different amino acid residue. In some embodiments, the at least one CDR of the constructs disclosed herein includes a sequence having 100% identity to the sequence of SEQ ID NO: 6, wherein at least 1, at least 2, at least 3, at least 4, at least 5 amino acid residues in the sequence is substituted by a different amino acid residue. In some embodiments, the at least one CDR includes a sequence having 100% identity to a sequence selected from the group consisting of SEQ ID NOs: 1-106, wherein one, two, three, four, or five of the amino acid residues in the sequence is substituted by a different amino acid residue. In some embodiments, the at least one CDR includes a sequence having 100% identity to the sequence of SEQ ID NO: 18, wherein one, two, three, four, or five of the amino acid residues in the sequence is substituted by a different amino acid residue.

One of ordinary skill in the art will understand that binding affinity can generally be used as a measure of the strength of a non-covalent interaction between two molecules, e.g., an antibody or functional fragment thereof and an antigen. In some cases, binding affinity can be used to describe monovalent interactions (intrinsic activity). Binding affinity between two molecules may be quantified by determination of the dissociation constant (K_{D}). In turn, K_{D} can be determined by measurement of the kinetics of complex formation and dissociation using, e.g., the surface plasmon resonance (SPR) method (Biacore). The rate constants corresponding to the association and the dissociation of a monovalent complex are referred to as the association rate constants kₐ (or kₒₙ) and dissociation rate constant k_{d} (or k_{off}), respectively. K_{D} is related to kₐ and k_{d} through the equation K_{D}=k_{d}/kₐ. The value of the dissociation constant can be determined directly by well-known methods, and can be computed even for complex mixtures by methods such as those set forth in Caceci et al. (1984, Byte 9:340-362). For example, the K_{D} may be established using a double-filter nitrocellulose filter binding assay such as that disclosed by Wong & Lohman (1993, Proc. Natl. Acad. Sci. USA 90:5428-5432). Other standard assays to evaluate the binding ability of engineered antibodies of the present disclosure towards target antigens are known in the art, including for example, ELISAs, Western blots, RIAs, and flow cytometry analysis, and other assays exemplified elsewhere herein. The binding kinetics and binding affinity of the antibody also can be assessed by standard assays known in the art, such as Surface Plasmon Resonance (SPR), e.g. by using a Biacore™ system, or KinExA. In some embodiments, the binding affinity of a construct as disclosure herein for a target antigen can be assessed by the Scatchard method described by Frankel et al., Mol. Immunol, 16:101-106, 1979.

In some embodiments, the construct of the disclosure can be (a) a TCR; (b) an antibody; or (c) a functional derivative or fragment of (a) or (b). One skilled in the art upon will readily understand that the term “functional fragment thereof” or “functional derivative thereof” refers to a molecule having quantitative and/or qualitative biological activity in common with the wild-type molecule from which the fragment or derivative was derived. For example, a functional fragment or a functional derivative of an antibody is one which retains essentially the same ability to bind to the same epitope as the antibody from which the functional fragment or functional derivative was derived. For instance, an antibody capable of binding to an epitope may be truncated at the N-terminus and/or C-terminus, and the retention of its epitope binding activity assessed using assays known to those of skill in the art.

In some embodiments, the construct is a TCR construct including a TCR alpha chain and a TCR beta chain operably linked to each other. In some embodiments, the TCR alpha chain and the TCR beta chain are covalently linked to each other. In some embodiments, the TCR alpha chain and the TCR beta chain are linked to each other in a non-covalent fashion. In some embodiments, the TCR alpha chain and the TCR beta chain are covalently linked to each other via a polypeptide linker. In some embodiments, the polypeptide linker is a cleavable linker. In some embodiments, the polypeptide linker includes an autoproteolytic peptide. In some embodiments, the autoproteolytic peptide includes one or more autoproteolytic cleavage sites derived from calcium-dependent serine endoprotease (furin), a porcine teschovirus-1 2A (P2A), a foot-and-mouth disease virus (FMDV) 2A (F2A), an Equine Rhinitis A Virus (ERAV) 2A (E2A), a Thosca asigna virus 2A (T2A), a cytoplasmic polyhedrosis virus 2A (BmCPV2A), a Flacherie Virus 2A (BmIFV2A), or a combination thereof. In some embodiments, the TCR alpha chain and the TCR beta chain are covalently linked to each other via a P2A cleavage site. The present disclosure provides both single-chain TCR constructs and multiple-chain TCR constructs. In some embodiments, the TCR constructs of the disclosure may be provided as single chain α or β, or γ and δ, molecules, or alternatively as double chain constructs composed of both the α and β chain, or γ and δ chain.

In some embodiments, the TCR construct of the disclosure may be provided as a single-chain TCR (scTCR). A scTCR can include a polypeptide of a variable region of a first TCR chain (e.g., an alpha chain) and a polypeptide of an entire (full-length) second TCR chain (e.g., a beta chain), or vice versa. In some embodiments, the polypeptides are directly linked to one another. In some embodiments, the scTCR can optionally include one or more linkers which join the two or more polypeptides together. In some embodiments, the linker can be a synthetic compound linker such as, for example, a chemical cross-linking agent. Non-limiting examples of suitable cross-linking agents that are available on the market include N-hydroxysuccinimide (NHS), disuccinimidylsuberate (DSS), bis(sulfosuccinimidyl) suberate (BS3), dithiobis(succinimidylpropionate) (DSP), dithiobis(sulfosuccinimidylpropionate) (DTSSP), ethyleneglycol bis(succinimidylsuccinate) (EGS), ethyleneglycol bis(sulfosuccinimidylsuccinate) (sulfo-EGS), disuccinimidyl tartrate (DST), disulfosuccinimidyl tartrate (sulfo-DST), bis[2-(succinimidooxycarbonyloxy)ethyl]sulfone (BSOCOES), and bis[2-(sulfosuccinimidooxycarbonyloxy)ethyl]sulfone (sulfo-BSOCOES).

In some embodiments, the linker can be a peptide linker, which joins together two single chains, as described herein. In some embodiments, the length and amino acid composition of the peptide linker sequence can be optimized to vary the orientation and/or proximity of the polypeptides relative to one another to achieve a desired activity of the constructs (e.g., TCR constructs) as disclosed herein.

The construct according to the present disclosure can also be provided in the form of a multimeric complex, including at least two scTCR molecules, wherein the scTCR molecules are each fused to at least one biotin moiety, or other interconnecting molecule/linker, and wherein the scTCRs are interconnected by biotin-streptavidin interaction to allow the formation of said multimeric complex. Similar approaches known in the art for the generation of multimeric TCR are also contemplated and included in this disclosure. Accordingly, also provided are multimeric complexes of a higher order, comprising more than two scTCR of the disclosure.

Suitable methods of making fusion polypeptides are known in the art, and include, for example, recombinant methods. In some embodiments, the constructs, TCRs (and functional fragments and functional derivatives thereof), and polypeptides of the disclosure may be expressed as a single protein including a linker peptide linking the α chain and the β chain, and/or linking the γ chain and the δ chain. In this regard, the constructs, TCRs (and functional fragments and functional derivatives thereof), and polypeptides of the disclosure include the amino acid sequences of the variable regions of the TCR of the disclosure and can further include a linker peptide. In some embodiments, the linker peptide may advantageously facilitate the expression of a construct or a TCR (including functional fragments and functional derivatives thereof) in a host cell. In principle, the linker peptide may comprise any suitable amino acid sequence. Linker sequences for single chain TCR constructs are well known in the art. In some embodiments, such a single chain construct can further comprise one, or two, constant domain sequences. Upon expression of the construct including the linker peptide by a host cell, the linker peptide may also be cleaved, resulting in separated α and β chains, and separated γ and δ chain.

In some embodiments, the TCR constructs of the disclosure includes at least one TCR α or γ and/or TCR β or δ variable domain. Generally, they include both a TCR α variable domain and a TCR β variable domain, alternatively both a TCR γ variable domain and a TCR δ variable domain. In some embodiments, the TCR constructs include αβ/γδ heterodimers or may be in single chain format. In some embodiments, fuse in adoptive therapy, an αβ or γδ heterodimeric TCR may, for example, be transfected as full length chains having both cytoplasmic and transmembrane domains. If desired, an introduced disulfide bond between residues of the respective constant domains can be present.

In some embodiments, the TCR constructs of the disclosure are provided as single chain α or β, or γ and δ, molecules, or alternatively as double chain constructs composed of both the α and β chain, or γ and δ chain. Accordingly, in some embodiments, the TCR construct is a single-chain TCR construct including in its beta chain a CDR3β having at least 70%, for example, at least 75%, at least 80%, at least 85%, at least 90%, at least 95%, at least 96%, at least 97%, at least 98%, at least 99%, or at least 100% sequence identity to a sequence selected from SEQ ID NOs: 1-106. In additional or alternative embodiments, the TCR construct may further include a CDR1 and/or a CDR2 domain sequence. In some embodiments, the TCR constructs of the disclosure include at least one, preferably all three CDR sequences CDR1, CDR2 and CDR3.

In some embodiments, the TCR constructs of the disclosure are provided as double-chain constructs composed of both the α and β chain, or γ and δ chain. Accordingly, in some embodiments, the TCR constructs of the disclosure are provided as double-chain constructs comprising both the α and β chain, wherein its beta chain includes a CDR3β having at least 70%, for example, at least 75%, at least 80%, at least 85%, at least 90%, at least 95%, at least 96%, at least 97%, at least 98%, at least 99%, or at least 100% sequence identity to a sequence selected from SEQ ID NOs: 1-106. In some embodiments, the TCR constructs further include in its alpha chain a CDR3a sequence.

As outlined above, in some embodiments, the construct of the disclosure can be provided in the framework of an antibody construct or a functional fragment thereof, which specifically binds to the antigens described herein. The antibody construct can be any type of immunoglobulin that is known in the art. For instance, the antibody construct can be of any iso-type, e.g., IgA, IgD, IgE, IgG, IgM, etc. The antibody construct can be monoclonal or polyclonal. The antibody construct can be a naturally-occurring antibody, e.g., an antibody isolated and/or purified from a mammal, e.g., human cell. Alternatively, the antibody construct can be a genetically-engineered antibody, e.g., a humanized antibody or a chimeric antibody. The antibody construct can be in monomeric or polymeric form. In some embodiments, the construct disclosed herein is an antibody construct selected from the group consisting of an antigen-binding fragment (Fab), a single-chain variable fragment (scFv), a nanobody, a single domain antibody (sdAb), a V_{H} domain, a V_{L} domain, a V_{H}H domain, a diabody, or a functional fragment of any thereof.

### B. Nucleic Acids

In one aspect, provided herein are various nucleic acid molecules including nucleotide sequences encoding the constructs of the disclosure, including expression cassettes, and expression vectors containing these nucleic acid molecules operably linked to heterologous nucleic acid sequences such as, for example, regulator sequences which allow in vivo expression of the constructs in a host cell or ex-vivo cell-free expression system.

The terms “nucleic acid molecule” and “polynucleotide” are used interchangeably herein, and refer to both RNA and DNA molecules, including nucleic acid molecules comprising cDNA, genomic DNA, synthetic DNA, and DNA or RNA molecules containing nucleic acid analogs. A nucleic acid molecule can be double-stranded or single-stranded (e.g., a sense strand or an antisense strand). A nucleic acid molecule may contain unconventional or modified nucleotides. The terms “polynucleotide sequence” and “nucleic acid sequence” as used herein interchangeably refer to the sequence of a polynucleotide molecule. The polynucleotide and polypeptide sequences disclosed herein are shown using standard letter abbreviations for nucleotide bases and amino acids as set forth in 37 CFR § 1.82), which incorporates by reference WIPO Standard ST.25 (1998), Appendix 2, Tables 1-6.

Nucleic acid molecules of the present disclosure can be nucleic acid molecules of any length, including nucleic acid molecules that are generally between about 0.5 Kb and about 50 Kb, for example between about 0.5 Kb and about 20 Kb, between about 1 Kb and about 15 Kb, between about 2 Kb and about 10 Kb, or between about 5 Kb and about 25 Kb, for example between about 10 Kb to 15 Kb, between about 15 Kb and about 20 Kb, between about 5 Kb and about 20 Kb, about 5 Kb and about 10 Kb, or about 10 Kb and about 25 Kb. In some embodiments, the nucleic acid molecules of the disclosure are between about 1.5 Kb and about 50 Kb, between about 5 Kb and about 40 Kb, between about 5 Kb and about 30 Kb, between about 5 Kb and about 20 Kb, or between about 10 Kb and about 50 Kb, for example between about 15 Kb to 30 Kb, between about 20 Kb and about 50 Kb, between about 20 Kb and about 40 Kb, about 5 Kb and about 25 Kb, or about 30 Kb and about 50 Kb.

In some embodiments disclosed herein, the nucleic acid molecules of the disclosure include a nucleotide sequence encoding a construct including at least one complementary determining region (CDR) having at least 70%, 75%, 80%, 85%, 90%, 95%, 96%, 97%, 98%, 99%, or 100% sequence identity to a sequence selected from the group consisting of SEQ ID NOs: 1-106. In some embodiments, the construct is single-chain constructs or double-chain constructs. In some embodiments, the construct is selected from the group consisting of: (a) a TCR; (b) an antibody; and (c) a functional derivative or fragment of (a) or (b). In some embodiments, the construct is a TCR construct including a TCR alpha chain and a TCR beta chain operably linked to each other. In some embodiments, the construct is a TCR construct including in its beta chain a CDR3β having at least 70%, 75%, 80%, 85%, 90%, 95%, 96%, 97%, 98%, 99%, or 100% sequence identity to a sequence selected from SEQ ID NOs: 1-106. In some embodiments, the construct further includes in its alpha chain a CDR3α sequence.

In some embodiments, the nucleotide sequence is incorporated into an expression cassette or an expression vector. It will be understood that an expression cassette generally includes a construct of genetic material that contains coding sequences and enough regulatory information to direct proper transcription and/or translation of the coding sequences in a recipient cell, in vivo and/or ex vivo. Generally, the expression cassette may be inserted into a vector for targeting to a desired host cell and/or into an individual. As such, in some embodiments, an expression cassette of the disclosure include a coding sequence for the construct as disclosed herein, which is operably linked to expression control elements, such as a promoter, and optionally, any or a combination of other nucleic acid sequences that affect the transcription or translation of the coding sequence.

In some embodiments, the nucleotide sequence is incorporated into an expression vector. It will be understood by one skilled in the art that the term “vector” generally refers to a recombinant polynucleotide construct designed for transfer between host cells, and that may be used for the purpose of transformation, e.g., the introduction of heterologous DNA into a host cell. As such, in some embodiments, the vector can be a replicon, such as a plasmid, phage, or cosmid, into which another DNA segment may be inserted so as to bring about the replication of the inserted segment. In some embodiments, the expression vector can be an integrating vector.

In some embodiments, the expression vector can be a viral vector. As will be appreciated by one of skill in the art, the term “viral vector” is widely used to refer either to a nucleic acid molecule (e.g., a transfer plasmid) that includes virus-derived nucleic acid elements that typically facilitate transfer of the nucleic acid molecule or integration into the genome of a cell or to a viral particle that mediates nucleic acid transfer. Viral particles will typically include various viral components and sometimes also host cell components in addition to nucleic acid(s). The term viral vector may refer either to a virus or viral particle capable of transferring a nucleic acid into a cell or to the transferred nucleic acid itself. Viral vectors and transfer plasmids contain structural and/or functional genetic elements that are primarily derived from a virus. In some embodiments, the viral vector is a bacculorival vector, a retroviral vector, or a lentiviral vector. The term “retroviral vector” refers to a viral vector or plasmid containing structural and functional genetic elements, or portions thereof, that are primarily derived from a retrovirus. The term “lentiviral vector” refers to a viral vector or plasmid containing structural and functional genetic elements, or portions thereof, including LTRs that are primarily derived from a lentivirus, which is a genus of retrovirus.

Accordingly, also provided herein are vectors, plasmids, or viruses containing one or more of the nucleic acid molecules encoding any of the constructs disclosed herein. The nucleic acid molecules can be contained within a vector that is capable of directing their expression in, for example, a cell that has been transformed/transduced with the vector. Suitable vectors for use in eukaryotic and prokaryotic cells are known in the art and are commercially available, or readily prepared by a skilled artisan.

DNA vectors can be introduced into eukaryotic cells via conventional transformation or transfection techniques. Suitable methods for transforming or transfecting cells can be found in Sambrook et al. (2012, supra) and other standard molecular biology laboratory manuals, such as, calcium phosphate transfection, DEAE-dextran mediated transfection, transfection, microinjection, cationic lipid-mediated transfection, electroporation, transduction, scrape loading, ballistic introduction, nucleoporation, hydrodynamic shock, and infection.

Viral vectors that can be used in the disclosure include, for example, baculoviral vectors, retrovirus vectors, adenovirus vectors, and adeno-associated virus vectors, lentivirus vectors, herpes virus, simian virus 40 (SV40), and bovine papilloma virus vectors (see, for example, Gluzman (Ed.), Eukaryotic Viral Vectors, CSH Laboratory Press, Cold Spring Harbor, N.Y.). For example, a chimeric receptor as disclosed herein can be produced in a eukaryotic cell, such as a mammalian cells (e.g., COS cells, NIH 3T3 cells, or HeLa cells). These cells are available from many sources, including the American Type Culture Collection (Manassas, VA). In selecting an expression system, care should be taken to ensure that the components are compatible with one another. Artisans or ordinary skill are able to make such a determination. Furthermore, if guidance is required in selecting an expression system, skilled artisans may consult P. Jones, “Vectors: Cloning Applications”, John Wiley and Sons, New York, N.Y., 2009).

The nucleic acid molecules provided can contain naturally occurring sequences, or sequences that differ from those that occur naturally, but, due to the degeneracy of the genetic code, encode the same polypeptide, e.g., antibody. These nucleic acid molecules can consist of RNA or DNA (for example, genomic DNA, cDNA, or synthetic DNA, such as that produced by phosphoamidite-based synthesis), or combinations or modifications of the nucleotides within these types of nucleic acids. In addition, the nucleic acid molecules can be double-stranded or single-stranded (e.g., either a sense or an antisense strand).

The nucleic acid molecules are not limited to sequences that encode polypeptides (e.g., antibodies); some or all of the non-coding sequences that lie upstream or downstream from a coding sequence (e.g., the coding sequence of a chimeric receptor) can also be included. Those of ordinary skill in the art of molecular biology are familiar with routine procedures for isolating nucleic acid molecules. They can, for example, be generated by treatment of genomic DNA with restriction endonucleases, or by performance of the polymerase chain reaction (PCR). In the event the nucleic acid molecule is a ribonucleic acid (RNA), molecules can be produced, for example, by in vitro transcription.

In another aspect, provided herein are cell cultures including at least one engineered cell as disclosed herein, and a culture medium. Generally, the culture medium can be any suitable culture medium for culturing the cells described herein. Techniques for transforming a wide variety of the above-mentioned cells and species are known in the art and described in the technical and scientific literature. Accordingly, cell cultures including at least one engineered cell as disclosed herein are also within the scope of this application. Methods and systems suitable for generating and maintaining cell cultures are known in the art.

### C. Engineered Cells and Cell Cultures

The recombinant nucleic acids of the present disclosure can be introduced into a cell, such as, for example, a human T lymphocyte, to produce an engineered cell containing the nucleic acid molecule. Accordingly, some embodiments of the disclosure relate to methods for making an engineered cell, including (a) providing a host cell capable of protein expression; and transducing the provided host cell with a recombinant nucleic acid of the disclosure to produce an engineered cell. Introduction of the nucleic acid molecules of the disclosure into cells can be achieved by methods known to those skilled in the art such as, for example, viral infection, transfection, conjugation, protoplast fusion, lipofection, electroporation, nucleofection, calcium phosphate precipitation, polyethyleneimine (PEI)-mediated transfection, DEAE-dextran mediated transfection, liposome-mediated transfection, particle gun technology, calcium phosphate precipitation, direct micro-injection, nanoparticle-mediated nucleic acid delivery, and the like.

Accordingly, in some embodiments, the nucleic acid molecules can be introduced into a host cell by viral or non-viral delivery vehicles known in the art to produce an engineered cell. For example, the nucleic acid molecule can be stably integrated in the engineered cell's genome, or can be episomally replicating, or present in the engineered cell as a mini-circle expression vector for transient expression. Accordingly, in some embodiments, the nucleic acid molecule is maintained and replicated in the recombinant host cell as an episomal unit. In some embodiments, the nucleic acid molecule is present in the engineered cell as a mini-circle expression vector for transient expression. In some embodiments, the nucleic acid molecule is stably integrated into the genome of the engineered cell. Stable integration can be achieved using classical random genomic recombination techniques or with more precise techniques such as guide RNA-directed CRISPR/Cas9 genome editing, or DNA-guided endonuclease genome editing with NgAgo (Natronobacterium gregoryi Argonaute), or TALENs genome editing (transcription activator-like effector nucleases).

The nucleic acid molecules can be encapsulated in a viral capsid or a lipid nanoparticle, or can be delivered by viral or non-viral delivery means and methods known in the art, such as electroporation. For example, introduction of nucleic acids into cells may be achieved by viral transduction. In a non-limiting example, baculoviral virus or adeno-associated virus (AAV) can be engineered to deliver nucleic acids to target cells via viral transduction. Several AAV serotypes have been described, and all of the known serotypes can infect cells from multiple diverse tissue types. AAV is capable of transducing a wide range of species and tissues in vivo with no evidence of toxicity, and it generates relatively mild innate and adaptive immune responses.

Lentiviral-derived vector systems are also useful for nucleic acid delivery and gene therapy via viral transduction. Lentiviral vectors offer several attractive properties as gene-delivery vehicles, including: (i) sustained gene delivery through stable vector integration into host genome; (ii) the capability of infecting both dividing and non-dividing cells; (iii) broad tissue tropisms, including important gene- and cell-therapy-target cell types; (iv) no expression of viral proteins after vector transduction; (v) the ability to deliver complex genetic elements, such as polycistronic or intron-containing sequences; (vi) a potentially safer integration site profile; and (vii) a relatively easy system for vector manipulation and production.

In some embodiments, host cells can be genetically engineered (e.g., transduced or transformed or transfected) with, for example, a vector construct of the present disclosure that can be, for example, a viral vector or a vector for homologous recombination that includes nucleic acid sequences homologous to a portion of the genome of the host cell, or can be an expression vector for the expression of the polypeptides of interest. Host cells can be either untransformed cells or cells that have already been transfected with at least one nucleic acid molecule.

In some embodiments, the engineered cell is a prokaryotic cell or a eukaryotic cell. In some embodiments, the cell is in vivo. In some embodiments, the cell is ex vivo. In some embodiments, the cell is in vitro. In some embodiments, the engineered cell is a eukaryotic cell. In some embodiments, the engineered cell is an animal cell. In some embodiments, the animal cell is a mammalian cell. In some embodiments, the animal cell is a human cell. In some embodiments, the cell is a non-human primate cell. In some embodiments, the engineered cell is an immune system cell, e.g., a B cell, a monocyte, a NK cell, a natural killer T (NKT) cell, a basophil, an eosinophil, a neutrophil, a dendritic cell, a macrophage, a regulatory T cell, a helper T cell (T_{H}), a cytotoxic T cell (T_{CTL}), a memory T cell, a gamma delta (γδ) T cell, another T cell, a hematopoietic stem cell, or a hematopoietic stem cell progenitor.

In some embodiments, the immune system cell is a lymphocyte. In some embodiments, the lymphocyte is a T lymphocyte. In some embodiments, the lymphocyte is a T lymphocyte progenitor. In some embodiments, the T lymphocyte is a CD4+ T cell or a CD8+ T cell. In some embodiments, the T lymphocyte is a CD8+ T cytotoxic lymphocyte cell. Non-limiting examples of CD8+ T cytotoxic lymphocyte cell suitable for the compositions and methods disclosed herein include naïve CD8+ T cells, central memory CD8+ T cells, effector memory CD8+ T cells, effector CD8+ T cells, CD8+ stem memory T cells, and bulk CD8+ T cells. In some embodiments, the T lymphocyte is a CD4+ T helper lymphocyte cell. Suitable CD4+T helper lymphocyte cells include, but are not limited to, naïve CD4+ T cells, central memory CD4+ T cells, effector memory CD4+ T cells, effector CD4+ T cells, CD4+ stem memory T cells, and bulk CD4+ T cells.

As outlined above, some embodiments of the disclosure relate to various methods for making an engineered cell of the disclosure, the methods include: (a) providing a host cell capable of protein expression; and transducing the provided host cell with a recombinant nucleic acid of the disclosure to produce an engineered cell. Non-limiting exemplary embodiments of the disclosed methods for making an engineered cell can further include one or more of the following features. In some embodiments, the cell is obtained by leukapheresis performed on a sample obtained from a subject, and the cell is transduced ex vivo. In some embodiments, the recombinant nucleic acid is encapsulated in a viral capsid or a lipid nanoparticle. In some embodiments, the methods further include isolating and/or purifying the produced cells. Accordingly, the engineered cells produced by the methods disclosed herein are also within the scope of the disclosure.

In another aspect, provided herein are cell cultures including at least one engineered cell as disclosed herein, and a culture medium. Generally, the culture medium can be any suitable culture medium for culturing the cells described herein. Techniques for transforming a wide variety of the above-mentioned cells and species are known in the art and described in the technical and scientific literature. Accordingly, cell cultures including at least one engineered cell as disclosed herein are also within the scope of this application. Methods and systems suitable for generating and maintaining cell cultures are known in the art.

### E. Pharmaceutical Compositions

The constructs, nucleic acids, engineered cells, and/or cell cultures of the disclosure can be incorporated into compositions, including pharmaceutical compositions. Such compositions generally include one or more of the constructs, nucleic acids, engineered cells, and/or cell cultures as provided and described herein, and a pharmaceutically acceptable excipient, e.g., carrier. In some embodiments, the pharmaceutical compositions of the disclosure are formulated for the treating, preventing, ameliorating a disease such as cancer, or for reducing or delaying the onset of the disease.

Accordingly, one aspect of the present disclosure relates to pharmaceutical compositions that include a pharmaceutically acceptable carrier and one or more of the following: (a) a construct of the disclosure; (b) a recombinant nucleic acid of the disclosure; and (c) an engineered cell of the disclosure. In some embodiments, the pharmaceutical compositions include (a) a construct of the disclosure and (b) a pharmaceutically acceptable carrier. In some embodiments, the pharmaceutical compositions include (a) a recombinant nucleic acid of the disclosure and (b) a pharmaceutically acceptable carrier. In some embodiments, the recombinant nucleic acid is encapsulated in a viral capsid or a lipid nanoparticle. In some embodiments, the pharmaceutical compositions of the disclosure include (a) an engineered cell of the disclosure and (b) a pharmaceutically acceptable carrier.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™. (BASF, Parsippany, N.J.), or phosphate buffered saline (PBS). In all cases, the composition should be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants, e.g., sodium dodecyl sulfate. Prevention of the action of microorganisms can be achieved by various antibacterial and antifungal agents, for example, parabens, chlorobutanol, phenol, ascorbic acid, thimerosal, and the like. In many cases, it will be generally to include isotonic agents, for example, sugars, polyalcohols such as mannitol, sorbitol, and/or sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

### Methods of the Disclosure

Administration of any one of the therapeutic compositions described herein, e.g., constructs, nucleic acids, engineered cells, and pharmaceutical compositions, can be used to treat subjects in the treatment of relevant diseases, such as cancers, immune diseases, and chronic infections. In some embodiments, the constructs, nucleic acids, engineered cells, and pharmaceutical compositions as described herein can be incorporated into therapeutic agents for use in methods of preventing and/or treating a subject who has, who is suspected of having, or who may be at high risk for developing one or more health conditions, such as proliferative disorders or microbial infections. Exemplary proliferative disorders can include, without limitation, angiogenic diseases, a metastatic diseases, tumorigenic diseases, neoplastic diseases and cancers. In some embodiments, the proliferatieve disorder is a cancer.

Accordingly, in one aspect, some embodiments of the disclosure relate to methods for the prevention and/or treatment of a condition in a subject in need thereof, wherein the methods include administering to the subject a composition including one or more of: (a) a construct of the disclosure; (b) a recombinant nucleic acid of the disclosure; (c) an engineered cell of the disclosure; and d) a pharmaceutically composition of the disclosure. In some embodiments, the composition includes a therapeutically effective amount or number of: (a) a construct of the disclosure; (b) a recombinant nucleic acid of the disclosure; (c) an engineered cell of the disclosure; and/or a pharmaceutical composition of the disclosure.

In some embodiments, the disclosed pharmaceutical composition is formulated to be compatible with its intended route of administration. The recombinant polypeptides of the disclosure may be given orally or by inhalation, but it is more likely that they will be administered through a parenteral route. Examples of parenteral routes of administration include, for example, intravenous, intradermal, subcutaneous, transdermal (topical), transmucosal, and rectal administration. Solutions or suspensions used for parenteral application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates and agents for the adjustment of tonicity such as sodium chloride or dextrose. pH can be adjusted with acids or bases, such as mono- and/or di-basic sodium phosphate, hydrochloric acid or sodium hydroxide (e.g., to a pH of about 7.2-7.8, e.g., 7.5). The parenteral preparation can be enclosed in ampoules, disposable syringes or multiple dose vials made of glass or plastic.

Dosage, toxicity and therapeutic efficacy of such subject recombinant polypeptides of the disclosure can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit high therapeutic indices are generally suitable. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the disclosure, the therapeutically effective dose can be estimated initially from cell culture assays. A dose may be formulated in animal models to achieve a circulating plasma concentration range that includes the IC₅₀ (e.g., the concentration of the test compound which achieves a half-maximal inhibition of symptoms) as determined in cell culture. Such information can be used to more accurately determine useful doses in humans. Levels in plasma may be measured, for example, by high performance liquid chromatography.

The therapeutically effective amount of a subject recombinant polypeptide of the disclosure (e.g, an effective dosage) depends on the polypeptide selected. For instance, single dose amounts in the range of approximately 0.001 to 0.1 mg/kg of patient body weight can be administered; in some embodiments, about 0.005, 0.01, 0.05 mg/kg may be administered. In some embodiments, 600,000 IU/kg is administered (IU can be determined by a lymphocyte proliferation bioassay and is expressed in International Units (IU). The compositions can be administered one from one or more times per day to one or more times per week; including once every other day. The skilled artisan will appreciate that certain factors may influence the dosage and timing required to effectively treat a subject, including but not limited to the severity of the disease, previous treatments, the general health and/or age of the subject, and other diseases present. Moreover, treatment of a subject with a therapeutically effective amount of the subject recombinant polypeptides of the disclosure can include a single treatment or, can include a series of treatments. In some embodiments, the compositions are administered every 8 hours for five days, followed by a rest period of 2 to 14 days, e.g., 9 days, followed by an additional five days of administration every 8 hours.

### Administration of Engineered Cells to a Subject

In some embodiments, the methods of treatment as disclosed herein involve administering an effective amount or number of the engineered cells to a subject in need of such treatment. This administering step can be accomplished using any method of implantation delivery in the art. For example, the engineered cells can be infused directly in the individual's bloodstream or otherwise administered to the individual.

In some embodiments, the methods disclosed herein include administering, which term is used interchangeably with the terms “introducing,” implanting,” and “transplanting,” engineered cells into a subjet, by a method or route that results in at least partial localization of the introduced cells at a desired site such that a desired effect(s) is/are produced. The engineered cells or their differentiated progeny can be administered by any appropriate route that results in delivery to a desired location in the individual where at least a portion of the administered cells or components of the cells remain viable. The period of viability of the cells after administration to a subjet can be as short as a few hours, e.g., twenty-four hours, to a few days, to as long as several years, or even the lifetime of the individual, i.e., long-term engraftment.

When provided prophylactically, the engineered cells described herein can be administered to a subjet in advance of any symptom of a disease or condition to be treated. Accordingly, in some embodiments the prophylactic administration of an engineered cell population prevents the occurrence of symptoms of the disease or condition.

When provided therapeutically in some embodiments, engineered cells are provided at (or after) the onset of a symptom or indication of a disease or condition, e.g., upon the onset of disease or condition.

For use in the various embodiments described herein, an effective amount or number of engineered cells as disclosed herein, can be at least 10² cells, at least 5×10² cells, at least 10³ cells, at least 5×10³ cells, at least 10⁴ cells, at least 5×10⁴ cells, at least 10⁵ cells, at least 2×10⁵ cells, at least 3×10⁵ cells, at least 4×10⁵ cells, at least 5×10⁵ cells, at least 6×10⁵ cells, at least 7×10⁵ cells, at least 8×10⁵ cells, at least 9×10⁵ cells, at least 1×10⁶ cells, at least 2×10⁶ cells, at least 3×10⁶ cells, at least 4×10⁶ cells, at least 5×10⁶ cells, at least 6×10⁶ cells, at least 7×10⁶ cells, at least 8×10⁶ cells, at least 9×10⁶ cells, or multiples thereof. The engineered cells can be derived from one or more donors or can be obtained from an autologous source. In some embodiments, the engineered cells are expanded in culture prior to administration to a subject in need of a treatment.

In some embodiments, the delivery of an engineered cell composition (e.g., a composition including a plurality of engineered cells according to any of the cells described herein) into an individual by a method or route results in at least partial localization of the cell composition at a desired site. A composition including engineered cells can be administered by any appropriate route that results in effective treatment in the individual, e.g., administration results in delivery to a desired location in the individual where at least a portion of the composition delivered, e.g., at least 1×10³ cells, is delivered to the desired site for a period of time. Modes of administration include injection, infusion, and instillation. “Injection” includes, without limitation, intravenous, intramuscular, intra-arterial, intrathecal, intraventricular, intracapsular, intraorbital, intracardiac, intradermal, intraperitoneal, transtracheal, subcutaneous, subcuticular, intraarticular, subcapsular, subarachnoid, intraspinal, intracerebrospinal, and intrasternal injection and infusion. In some embodiments, the route is intravenous. For the delivery of cells, delivery by injection or infusion is a standard mode of administration.

In some embodiments, the engineered cells are administered systemically, e.g., via infusion or injection. For example, a population of engineered cells are administered other than directly into a target site, tissue, or organ, such that it enters the individual's circulatory system and, thus, is subject to metabolism and other similar biological processes.

The efficacy of a treatment including any of the compositions provided herein for the treatment of a disease or condition can be determined by a skilled clinician. However, one skilled in the art will appreciate that a treatment is considered effective if any one or all of the signs or symptoms or markers of disease are improved or ameliorated. Efficacy can also be measured by failure of a subject to worsen as assessed by decreased hospitalization or need for medical interventions (e.g., progression of the disease is halted or at least slowed). Methods of measuring these indicators are known to those of skill in the art and/or described herein. Treatment includes any treatment of a disease in a subject or an animal (some non-limiting examples include a human, or a mammal) and includes: (1) inhibiting the disease, e.g., arresting, or slowing the progression of symptoms; or (2) relieving the disease, e.g., causing regression of symptoms; and (3) preventing or reducing the likelihood of the development of symptoms.

As discussed above, a therapeutically effective number of engineered cells refers to a number of engineered cells that is sufficient to promote a provide a therapeutic benefit in the treatment or management of a disease, e.g., cancer, or to delay or minimize one or more symptoms associated with the disease when administered to a subject, such as one who has, is suspected of having, or is at risk for the disease. In some embodiments, an effective number includes a number sufficient to prevent or delay the development of a symptom of the disease, alter the course of a symptom of the disease (for example but not limited to, slow the progression of a symptom of the disease), or reverse a symptom of the disease. In some embodiments, an effective number includes a number sufficient to inhibit tumor growth or metastasis of a cancer in the individual. In some embodiments, an effective number includes a number sufficient to increase cytokine production, inhibit (e.g., kill) a cancer cell or an infected cell.

In some embodiments of the disclosed methods, the individual is a mammal. In some embodiments, the mammal is a human. In some embodiments, the individual has or is suspected of having a condition associated with a proliferative disorder or disease, such as a cancer. The term cancer generally refers to the presence of cells possessing characteristics typical of cancer-causing cells, such as uncontrolled proliferation, immortality, metastatic potential, rapid growth and proliferation rate, and certain characteristic morphological features. Cancer cells are often observed aggregated into a tumor, but such cells can exist alone within an animal subject, or can be a non-tumorigenic cancer cell, such as a leukemia cell. Thus, the terms “cancer” or can encompass reference to a solid tumor, a soft tissue tumor, or a metastatic lesion. As used herein, the term “cancer” includes premalignant, as well as malignant cancers. In some embodiments, the cancer is a solid tumor, a soft tissue tumor, or a metastatic lesion.

Examples of conditions suitable for being treated by the compositions and methods of the disclosure include those associated with cancers, autoimmune diseases, inflammatory diseases, and infectious diseases. In some embodiments, the proliferative disorder is a cancer. Examples of cancers that can be suitably diagnosed, prevented, and/or treated by the compositions and methods of the disclosure include lung cancers. In principle, there are no particular limitations to the in regard to the lung cancers that can be suitably diagnosed, prevented, and/or treated by the compositions and methods disclosed herein. Examples of suitable lung cancers include adenocarcinoma, squamous cell carcinoma, small cell carcinoma, non-small cell carcinoma, adenosquamous carcinoma, small cell lung cancer, large cell carcinoma, neuroendocrine cancers of the lung, non-small cell lung cancer (NSCLC). Additional lung cancers that can be suitably diagnosed, prevented, and/or treated by the compositions and methods disclosed herein include, but are not limited to, undifferentiated non-small cell carcinoma, non-small cell carcinoma not otherwise specified, pulmonary squamous cell carcinoma, broncho-alveolar carcinoma, sarcomatoid carcinoma, pleomorphic carcinoma, carcinosarcoma, pulmonary blastoma, metastatic carcinoma of unknown primary, primary pulmonary lymphoepithelioma-like carcinoma, and benign neoplasms of the lung. In some embodiments, the cancer is NSCLC. In some embodiments, the lung cancer is a NSCLC selected from the group consisting of squamous cell carcinoma, adenocarcinoma, large cell carcinoma, carcinoid tumor, pleomorphic, salivary gland cancer, adenosquamous, sarcomatoid, and unclassified carcinomas. In some embodiments, the NSCLC is squamous cell carcinoma. In some embodiments, the NSCLC is adenocarcinoma. In some embodiments, the NSCLC is large cell carcinoma. In some embodiments, the NSCLC includes stage I NSCLC. In some embodiments, the NSCLC includes stage II NSCLC.

In some embodiments, the cancer is a multiply drug resistant cancer or a recurrent cancer. It is contemplated that the compositions and methods disclosed here are suitable for both non-metastatic cancers and metastatic cancers. Accordingly, in some embodiments, the cancer is a non-metastatic cancer. In some other embodiments, the cancer is a metastatic cancer. In some embodiments, the composition administered to the subject inhibits metastasis of the cancer in the subject. In some embodiments, the administered composition inhibits tumor growth in the subject.

In another aspect, provided herein are methods for assisting in the prevention and/or treatment of a condition in a subject in need thereof, the methods including the steps of administering to the subject a first therapy including one or more constructs, recombinant nucleic acids, engineered cells, or pharmaceutical compositions as disclosed herein, and administering to the subject at least one additional therapies, wherein the first therapy and at least one additional therapies together prevent and/or treat the condition in the subject. In some embodiments, the methods include administering to the subject a first therapy including an effective number of the engineered cells as disclosed herein, wherein the engineered cells treat the condition.

Additional examples of conditions suitable for being treated by the compositions and methods of the disclosure include those associated with an immune checkpoint blockade. In these instances, the method of the disclosure may be for an immunotherapy of a checkpoint blockade. In some embodiments, the checkpoint blockade immunotherapy involves using one or more inhibitors of a checkpoint receptor such as, for example, PD-1/PD-L1, CTLA-4, IDO, TIM3, LAG3, TIGIT, BTLA, VISTA, ICOS, KIRs and CD39. In some embodiments, the checkpoint receptor is an inhibitory checkpoint receptor selected from the group consisting of PD-1, CTLA-4, A2AR, B7-H3, B7-H4, BTLA, CD5, CD132, IDO, KIR, LAG3, TIM-3, TIGIT, VISTA. In some embodiments, the checkpoint receptor is a stimulatory checkpoint receptor selected from the group consisting of CD27, CD28, CD40, OX40, GITR, ICOS, and CD137. In some embodiments, the checkpoint blockade immunotherapy includes an anti-PD1 checkpoint therapy. In some embodiments, the checkpoint blockade immunotherapy includes an anti-PD1-L1 checkpoint therapy.

As described in greater detail below, various constructs of the disclosure are capable of binding antigens derived from viral pathogens. In some embodiments of the disclosure, provided herein are methods for the diagnosis, prevention, and/or treatment of a malignancy associated with a microbial infection. In some embodiments, the malignancy is associated with a bacterial infection.

In some embodiments, provided herein are methods for the diagnosis, prevention, and/or treatment of a malignancy associated with a viral infection. In some embodiments, the malignancy associated with an infection by Epstein-Barr virus (EBV), which was originally discovered through its association with Burkitt lymphoma, but has since been linked to a remarkably wide range of lymphoproliferative lesions and malignant lymphomas of B-, T- and NK-cell origin. Examples of EBV-associated malignancies that can suitably be diagnosed, prevented, and/or treated by using the compositions and methods disclosed herein include Hodgkin lymphoma, Burkitt lymphoma, diffuse large B cell lymphoma, nasopharyngeal carcinoma, gastric carcinoma, post-transplant lymphoproliferative disease, B lymphoproliferative disease. Additional EBV-associated malignancies that can suitably be diagnosed, prevented, and/or treated by using the compositions and methods disclosed herein include, but are not limited to, T-cell lymphoproliferative disease, NK-cell lymphoproliferative disease, NK-cell lymphomas, T-cell lymphomas, NK-cell lymphomas, T-cell leukemias, leiomyosarcomas, and lymphoepithelioma-like carcinomas.

### Additional Therapies

As discussed above, some embodiments of the disclosure provide methods for the prevention or treatment of a condition in a subject, wherein the methods include administering a composition as disclosed herein to the subject as a single therapy (e.g., monotherapy). In addition, in some embodiments of the disclosure, the composition is administered to the subject individually as a first therapy or in combination with at least one additional therapies, e.g., at least one, two, three, four, or five additional therapies. Suitable therapies to be administered in combination with the compositions of the disclosure include, but are not limited to chemotherapy, radiotherapy, immunotherapy, hormonal therapy, toxin therapy, targeted therapy, and surgery.

Non-limiting examples of therapies suitable for combining with the methods disclosed herein include is an anti-CTLA4 antibody, an anti-PD-1 antibody, an anti-PD-L1 antibody, an anti-CD20 antibody, an anti-CD40 antibody, an anti-DR5 antibody, an anti-CD1d antibody, an anti-TIM3 antibody, an anti-SLAMF7 antibody, an anti-KIR receptor antibody, an anti-OX40 antibody, an anti-HER2 antibody, an anti-ErbB-2 antibody, an anti-EGFR antibody, cetuximab, rituximab, trastuzumab, pembrolizumab. Additional therapies suitable for combining with the methods disclosed herein include, but are not limited to, radiotherapy such as single dose radiation, fractionated radiation, and focal radiation, and whole organ radiation. Also suitable for combining with the methods disclosed herein include IL-12, IFNα, GM-CSF, chimeric antigen receptors, adoptively transferred T cells, anti-cancer vaccines, and oncolytic viruses.

In some embodiments, the first therapy and the at least one additional therapies are administered concomitantly. In some embodiments, the first therapy is administered at the same time as the at least one additional therapies. In some embodiments, the first therapy and the at least one additional therapies are administered sequentially. In some embodiments, the first therapy is administered before the at least one additional therapies. In some embodiments, the first therapy is administered after the at least one additional therapies. In some embodiments, the first therapy is administered before and/or after the at least one additional therapies. In some embodiments, the first therapy and the at least one additional therapies are administered in rotation. In some embodiments, the first therapy and the at least one additional therapies are administered together in a single formulation.

In yet another aspects, provided herein are various methods for obtaining a construct as disclosed herein, the methods include (a) identifying a plurality of TCRs associated with a health condition; (b) determining a sequence of a CDR3β present in each of the identified TCRs; and making a construct including a CDR3β sequence determined in (b). In some embodiments, the methods further include identifying one or more cognate antigens commonly recognized by the CDR3β sequences. In some embodiments, the condition is associated with a proliferative disease. In some embodiments, the proliferative disease is a cancer. In some embodiments, the cancer is a lung cancer. In some embodiments, the condition is a malignancy associated with a viral infection. In some embodiments, the condition is a malignancy associated with an infection by Epstein-Barr virus (EBV). In some embodiments, the condition is associated with an immune checkpoint blockade.

### Kits

Also provided herein are various kits for the practice of a method described herein. In particular, some embodiments of the disclosure provide kits for the diagnosis of a condition in a subject. Some other embodiments relate to kits for the prevention of a condition in a subject in need thereof. Some other embodiments relate to kits for methods of treating a condition in a subject in need thereof. For example, provided herein, in some embodiments, are kits that include one or more of the constructs, recombinant nucleic acids, engineered cells, or pharmaceutical compositions as provided and described herein, as well as written instructions for making and using the same.

In some embodiments, the kits of the disclosure further include one or more means useful for the administration of any one of the provided constructs, recombinant nucleic acids, engineered cells, or pharmaceutical compositions to an individual. For example, in some embodiments, the kits of the disclosure further include one or more syringes (including pre-filled syringes) and/or catheters (including pre-filled syringes) used to administer any one of the provided constructs, recombinant nucleic acids, engineered cells, or pharmaceutical compositions to an individual. In some embodiments, a kit can have one or more additional therapeutic agents that can be administered simultaneously or sequentially with the other kit components for a desired purpose, e.g., for diagnosing, preventing, or treating a condition in a subject in need thereof.

Any of the above-described kits can further include one or more additional reagents, where such additional reagents can be selected from: dilution buffers; reconstitution solutions, wash buffers, control reagents, control expression vectors, negative control constructs, positive control constructs, and reagents suitable for in vitro production of the constructs.

In some embodiments, the components of a kit can be in separate containers. In some other embodiments, the components of a kit can be combined in a single container.

In some embodiments, a kit can further include instructions for using the components of the kit to practice the methods disclosed herein. For example, the kit can include a package insert including information concerning the pharmaceutical compositions and dosage forms in the kit. Generally, such information aids patients and physicians in using the enclosed pharmaceutical compositions and dosage forms effectively and safely. For example, the following information regarding a combination of the disclosure may be supplied in the insert: pharmacokinetics, pharmacodynamics, clinical studies, efficacy parameters, indications and usage, contraindications, warnings, precautions, adverse reactions, overdosage, proper dosage and administration, how supplied, proper storage conditions, references, manufacturer/distributor information and intellectual property information.

The instructions for practicing the methods are generally recorded on a suitable recording medium. For example, the instructions can be printed on a substrate, such as paper or plastic, etc. The instructions can be present in the kit as a package insert, in the labeling of the container of the kit or components thereof (e.g., associated with the packaging or sub-packaging), etc. The instructions can be present as an electronic storage data file present on a suitable computer readable storage medium, e.g. CD-ROM, diskette, flash drive, etc. In some instances, the actual instructions are not present in the kit, but means for obtaining the instructions from a remote source (e.g., via the internet), can be provided. An example of this embodiment is a kit that includes a web address where the instructions can be viewed and/or from which the instructions can be downloaded. As with the instructions, this means for obtaining the instructions can be recorded on a suitable substrate.

All publications and patent applications mentioned in this disclosure are herein incorporated by reference to the same extent as if each individual publication or patent application was specifically and individually indicated to be incorporated by reference.

No admission is made that any reference cited herein constitutes prior art. The discussion of the references states what their authors assert, and the Applicant reserves the right to challenge the accuracy and pertinence of the cited documents. It will be clearly understood that, although a number of information sources, including scientific journal articles, patent documents, and textbooks, are referred to herein; this reference does not constitute an admission that any of these documents forms part of the common general knowledge in the art.

The discussion of the general methods given herein is intended for illustrative purposes only. Other alternative methods and alternatives will be apparent to those of skill in the art upon review of this disclosure, and are to be included within the spirit and purview of this application.

Additional embodiments are disclosed in further detail in the following examples, which are provided by way of illustration and are not in any way intended to limit the scope of this disclosure or the claims.

### EXAMPLES

The practice of the present invention will employ, unless otherwise indicated, conventional techniques of molecular biology, microbiology, cell biology, biochemistry, nucleic acid chemistry, and immunology, which are well known to those skilled in the art. Such techniques are explained fully in the literature, such as Sambrook, J., & Russell, D. W. (2012). Molecular Cloning: A Laboratory Manual (4th ed.). Cold Spring Harbor, NY: Cold Spring Harbor Laboratory and Sambrook, J., & Russel, D. W. (2001). Molecular Cloning: A Laboratory Manual (3rd ed.). Cold Spring Harbor, NY: Cold Spring Harbor Laboratory (jointly referred to herein as “Sambrook”); Ausubel, F. M. (1987). Current Protocols in Molecular Biology. New York, NY: Wiley (including supplements through 2014); Bollag, D. M. et al. (1996). Protein Methods. New York, NY: Wiley-Liss; Huang, L. et al. (2005). Nonviral Vectors for Gene Therapy. San Diego: Academic Press; Kaplitt, M. G. et al. (1995). Viral Vectors: Gene Therapy and Neuroscience Applications. San Diego, CA: Academic Press; Lefkovits, I. (1997). The Immunology Methods Manual: The Comprehensive Sourcebook of Techniques. San Diego, CA: Academic Press; Doyle, A. et al. (1998). Cell and Tissue Culture: Laboratory Procedures in Biotechnology. New York, NY: Wiley; Mullis, K. B., Ferré, F. & Gibbs, R. (1994). PCR: The Polymerase Chain Reaction. Boston: Birkhauser Publisher; Greenfield, E. A. (2014). Antibodies: A Laboratory Manual (2nd ed.). New York, NY: Cold Spring Harbor Laboratory Press; Beaucage, S. L. et al. (2000). Current Protocols in Nucleic Acid Chemistry. New York, NY: Wiley, (including supplements through 2014); and Makrides, S. C. (2003). Gene Transfer and Expression in Mammalian Cells. Amsterdam, NL: Elsevier Sciences B. V., the disclosures of which are incorporated herein by reference.

### Example 1

### Clinical Samples

Protocols for collection of human tissue and blood were approved by the Stanford Institutional Review Board (IRB 15166 and IRB 21319). Inclusion criteria included adult patients (age >=18 years), known or suspected diagnosis of NSCLC, primary tumor >2 cm, and consent for research. Patients receiving neoadjuvant therapy or patients with underlying lung infection, inflammatory, or fibrotic disease were excluded. Overall 21 patients with surgically-resectable NSCLC treated at Stanford were included in this study. DNA was extracted from peripheral blood PBMC (Qiagen) for HLA tying.

### Example 2

### Tissue Processing

Tissue was processed within 2 hours from surgery. Tissue was divided and one section for cell suspensions and another section for histology. Cell suspensions were generated by mincing of tissue followed by digestion with collagenase III (200 IU/mL) and DNAse (100 U/mL) (Worthington Biochemical) for 40 minutes in RPMI and passing through a 70-um filter. Sections for histology were fixed in 4% paraformaldehyde and transferred to 70% ethanol solution the following day.

### Example 3

### FACS Analyses

T cells were isolated from tumor single cell suspensions by antibody staining followed by cell sorting on a 5-laser FACSAria Fusion (Stanford FACS Facility) purchased using funds from the Parker Institute for Cancer Immunotherapy. Tumor cell suspensions were stained in PBS with Zombie Aqua dye (Biolegend) for viability assessment. This was followed by staining in PBS with 2% FBS in Fc Blocking solution (Biolegend) plus the following antibodies: anti-CD4 (OKT4, Biolegend), anti-CD8 (SK1, Biolegend), anti-CD3 (OKT3, Biolegend), anti-CD45 (H130, Biolegend), anti-CD25 (BC96, Biolegend), anti-PD-1 (EH12.2H7, Biolegend), anti-CD137 (4B4-1, BD Biosciences), anti-HLA-DR (L243, Biolegend). CD3+CD45+AquaZombie-cells were index sorted directly into 96-well plates preloaded with 4 μL of capture buffer, snap frozen on dry ice, and stored at −80° C.

### Example 4

### GLIPH2 Analyses and Establishment of T Cell Specificity Groups

The GLIPH2 algorithm was implemented for the establishment of T cell specificity groups using 778,938 distinct CDR3β sequences from the MD Anderson NSCLC dataset [25]. Briefly, by comparing with the reference dataset of 273,920 distinct CDR3β sequences (both CD4 and CD8) from 12 healthy individuals, GLIPH2 first discovered clusters of CDR3β sequences sharing either global or local motifs as previously described [24]. The output of CDR3β clusters with shared sequence motifs is accompanied by multiple statistical measurements to facilitate the calling of high-confidence specificity groups, including biases in Vβ gene usage, CDR3β length distribution (relevant only for local motifs), cluster size, HLA allele usage, and clonal expansion. To establish high-confidence specificity groups with the NSCLC dataset, TCR specificity groups with at least 3 distinct CDR3β members from a minimum of 3 different patients with significant biases in Vβ gene usage, and CDR3β clonal expansion in comparison with the reference dataset were prioritized. This led to the discovery of 4,226 specificity groups that formed the basis for further analyses throughout the study.

### Example 5

Annotation of Specificity Groups with Tetramer-Derived CDR3β Sequences

To annotate inferred specificity groups from lung cancer patients, a combined GLIPH analysis using both the MD Anderson lung cancer patient CDR3β sequences and publicly available, tetramer-derived CDR3β sequences was performed. To do so, tetramer-derived CDR3β sequences that could form TCR specificity groups were first identified by running an independent GLIPH analysis with a total 10,051 CDR3β sequences from the tetramer datasets. This led to the formation of 395 specificity groups containing 1,561 CDR3β sequences. These 1,561 CDR3β sequences were then combined with the 778,938 CDR3β sequences from the MD Anderson lung cancer dataset for the aforementioned GLIPH2 analysis. Any specificity group that includes at least one CDR3β sequence from the tetramer data is considered “annotated” and would be assigned a specificity and HLA restriction according to the associated tetramer sequence(s). Of note, in all cases where multiple tetramer-derived CDR3β sequences were found in a given specificity group, there was only one dominant tetramer-defined specificity/HLA involved.

### Example 6

Single-Cell RNA-Seq (scRNA-Seq) Sample Preparation with the Smart-Seq2 Method

Full transcriptomes from FACS sorted T cells at the single-cell level were generated according to the previously reported procedures with some modifications [54]. First strand cDNA was then generated with Takara's SMARTScribe Reverse Transcriptase kit according to manufacturer's protocol (Takara Bio). Notable changes from the previously reported Smart-Seq2 RT step includes: 2 mM of dNTP and 2 μM of oligo-dT were included in the capture buffer; IM of Betaine and additional 6 mM MgCl2 were included in the RT reaction buffer. The cDNA samples were then amplified with the KAPA Library Quantification kit for 22-25 cycles (Roche). One microliter of amplified cDNA (of total 25/well) was used for single-cell TCR-sequencing and thus bypassing the RT step as reported previously [35]. To proceed with scRNA-Seq, full-length cDNA samples were first cleaned up with 0.6-0.8× volume of pre-calibrated AMPure XP beads (Beckman Coulter) to exclude DNA fragments smaller than 500 base pairs. The automatic liquid handler Biomek FXP Automated Workstation (Beckman Coulter) was used in order to eliminate cell-to-cell variabilities. The quality of purified full-length cDNA was validated with the AATI Fragment Analyzer (Agilent). Subsequently, the measurements from the Fragment Analyzer were used in order to normalize the cDNA input with a Mantis liquid handler (Formulatrix). The cDNA samples were then consolidated into a 384-well plate (LVSD) with a Mosquito X1 liquid handler (TTP labtech). After transfer, Illumina sequencing libraries were prepared using a Mosquito HTS liquid handler (TTP labtech). Only 0.4 uL (of total 23 uL) of cDNA per well were used to make the full transcriptome libraries with the Nextera XT DNA Library Preparation Kit (Illumina, FC-131-1096). Custom-made i5 and i7 unique 8-bp indexing primers (IDT) were used to multiplex 384 wells in a single sequencing run. The libraries were amplified on a C1000 Touch™ Thermal Cycler with 384-Well Reaction Module (Bio-rad). The pooled libraries were checked with the Agilent 2100 Bioanalyzer (Stanford PAN facility) and acquired paired-end sequences (150 bp×2) on a Hiseq 4000 Sequencing System (Illumina) purchased with funds from NIH (S10OD018220) for the Stanford Functional Genomics Facility (SFGF).

### Example 7

### Single-Cell Sequencing of the TCRα/β Chains

Single T cells were sorted and captured as described above in the method for scRNA-Seq sample preparation. Following first strand cDNA synthesis (Takara) and amplification (Roche), one microliter of amplified cDNA (of total 25 uL/well) was used for single-cell TCR-sequencing and thus bypassing the RT step as reported previously [35]. Nested PCR was performed with TCRα/β primers carrying multiplexing barcodes that enabled pooled CDR3α/β sequencing in a single Miseq run. Paired sequencing reads were joined, demultiplexed, and mapped to the human TCR references from the international ImMunoGeneTics information System® (IMGT) with custom scripts as reported previously.

### Example 8

Data Analyses of scRNA-Seq Results

Sequencing reads were first de-multiplexed and binned into separate fastq files that correspond with the full transcriptomes of individual T cells. STAR aligner (2.7.1a) was used to map the reads with default parameters against human genome reference GRCh38 (v21) from the UCSC genome browser. Mapped reads were sorted and indexed with samtools (1.4). Gene expression was first quantified by counting reads mapped to genes with htseq-count (HTSeq 0.9.1) using the following settings:—stranded=no—type=exon—idattr=gene_name—mode=intersection-nonempty. Unless otherwise stated, all single-cell T cell states were analyzed with Seurat (3.1.4) packages in R using raw read counts. To derive TCR repertoires from the scRNA-Seq results, reads mapped to both the TCRα and TCRβ genes were first reconstructed with the TraCeR algorithm as described previously [36]. The reconstructed DNA sequences were then submitted to the IMGT to call gene segment usage and the CDR3 amino acid sequences through HighV-QUEST.

### Example 9

GLIPH2 Analysis on the CDR3β Sequences from the TRACERx NSCLC Cohort

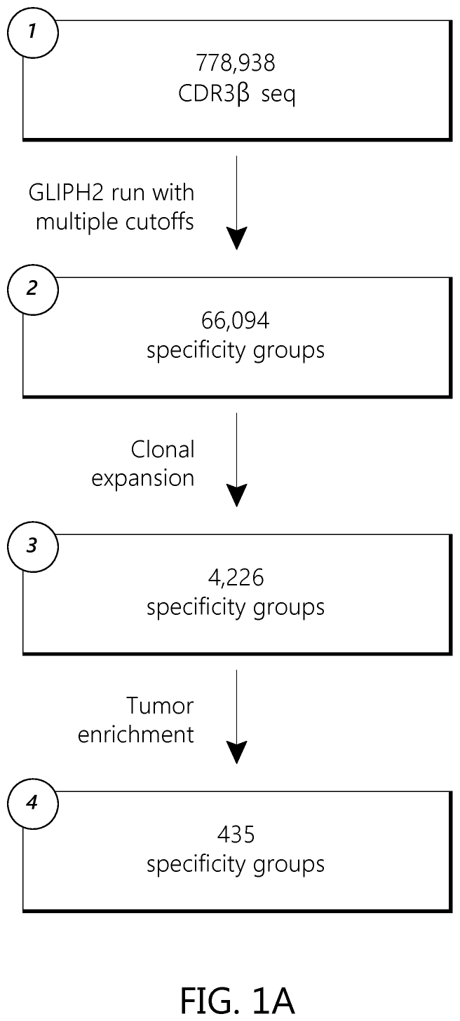

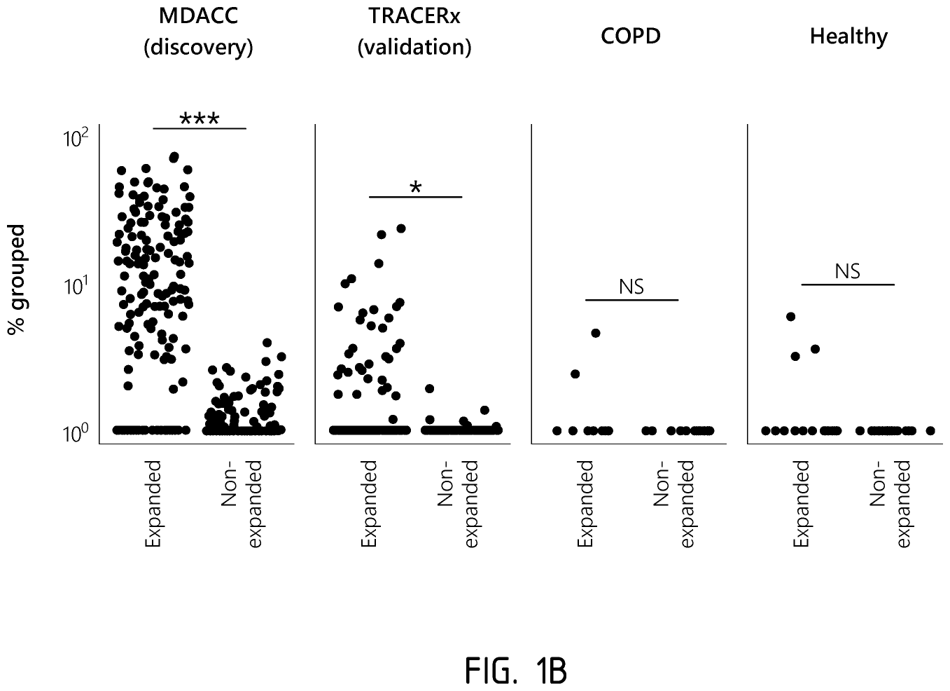

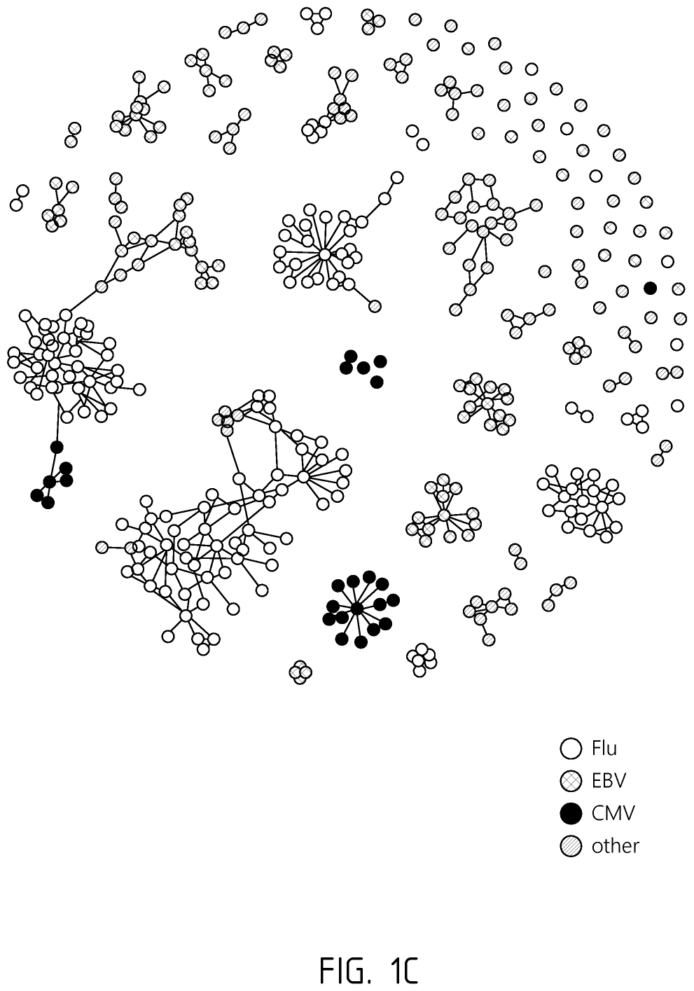

Raw fastq files (n=202) of the bulk CDR3β nucleotide sequences from the TRACERx cohort of NSCLC were downloaded from the Short Read Archive as reported [26]. The amino acid sequences of CDR3β, V gene usage, and clonal counts were subsequently derived by using the custom pipeline established previously [35]. To quantify the percentages of tumor-enriched specificity groups shown in FIG. 1B, joint GLIPH2 analyses were first conducted with combined CDR3β sequences from the MD Anderson cohort (n=778,938) and the bulk CDR3β sequences from each tumor sample of the TRACERx cohort. The total percentages (%) of top-20 clonally expanded as well as the rest CDR3β clonotypes that belonged to the 449 tumor-enriched specificity groups were then derived for each tumor (n=202).

### Example 10

### Soluble Biotinylated TCRα/β Chains for Yeast Screen

Soluble TCRα/β chains used for yeast selections were made as described previously [33]. Briefly, synthetic gene blocks (gBlocks®) of N-terminal truncated TCRα or TCRβ chain V and modified C gene fragments were assembled into the baculoviral pAcGP67a construct (BD Biosciences) with Gibson assembly (New England BioLabs). The final baculoviral plasmid was co-transfected into SF9 cells (ATCC) with Bestbac 2.0 (Expression systems) with FuGENER 6 (Promega) to make the crude viral supernatant (P0). Subsequently, viruses were passaged at a dilution of 1:500 in 30-50 mL cultures at a density of 1×10⁶ cells/mL to generate higher titer viruses (P1). To generate the soluble TCRα/β chains, up to 4 liters of High Five (Hi5, Thermo Fisher Scientific) cells were infected with PI baculovirus at a dilution of 1:500-1:1000 at a density of 2×10⁶ cells/mL for a week before protein purification. Recombinant TCRα/β chains were bound with Ni-NTA resin (QIAGEN) in the Hi5 cell media for 3 hours at room temperature, washed with 20 mM imidazole in 1× HBS at pH 7.2, and eluded eluted in 200 mM imidazole in 1× HBS at pH 7.2. After buffer exchange to 1× HBS at pH 7.2 with a 30 kDa filter (Millipore), purified proteins were biotinylated overnight with birA ligase in the presence of 100 μM biotin, 40 mM Bicine at pH 8.3, 10 mM ATP, and 10 mM Magnesium Acetate at 4° C. Biotinylated proteins were purified by size-exclusion chromatography using an AKTAPurifier Superdex 200 column (GE Healthcare) and validated on a SDS-PAGE gel to confirm the stoichiometry and biotinylation with excess streptavidin.

### Example 11

### Lentiviral TCR Transduction

TCRα chain, P2A linker, and TCRβ chain fusion gene fragments were purchased from IDT and cloned into MCS of the EF1α-MCS-GFP-PGK-puro lentiviral vector [23]. HEK-293T cells were plated on a 10-cm dish at a density of 7.5×10⁶ cells in 10 mL of DMEM the day prior to transfection. 293 Ts were co-transfected with 3.3 μg of the lentiviral plasmid, 2.5 μg of the gag-pol plasmid, and 0.83 μg of the VSV-G envelope plasmid pre-mixed with 33 μL of PEI in 120 μL of Opti-MEM (ThermoFisher Scientific). After 24 hours, the medium was replenished and viral supernatant was collected 24 and 48 hours later. TCR-deficient Jurkat cells (below) were transduced with viral supernatant, TCR expression was assessed by flow cytometry, and TCR-expressing cells were sorted based on the expression of GFP, CD3, and the transduced TCRα/β chains.

### Example 12

### Retroviral TCR Transduction

For retroviral-mediated expression of TCR27 and TCR28 in primary T cells, TCRα chain, P2A linker, and TCRβ chain were PCR amplified from the lentiviral vector (described above) and cloned into the MCS of an MSGV1-based retroviral vector (gift from Steve Rosenberg laboratory) using In-Fusion Cloning (Takara). For retroviral-mediated expression of TCR27 and TCR28 in primary T cells, TCRα chain, P2A linker, and TCRβ chain fusion gene fragments were purchased from IDT and cloned into MCS of an MSGV1-based retroviral vector.

### Example 13

### Cell Cultures

The Jurkat 76 T-cell line deficient for both TCRα and TCRβ were provided by Dr. Shao-An Xue (Department of Immunology, University of College London). Jurkat cells and primary T cells were grown in complete RPMI (ThermoFisher) containing 10% FBS, 25 mM HEPES, 290 μg/mL L-glutamine, 100 U/mL penicillin, 100 U/mL streptomycin, 1 mM sodium pyruvate, and 1× non-essential amino acids. T2 cells were grown in IMDM (Fisher Scientific) with 20% FBS, 290 μg/mL L-glutamine, 100 U/mL penicillin, 100 U/mL streptomycin.

### Example 14

### In Vitro Stimulation of the Jurkat T Cell Clones

Jurkat 76 cells expressing the exogenous TCR of interest were sorted and co-cultured with T2 cells in complete RPMI as detailed above. Peptides were dissolved in DMSO at 20 mM stock concentration and diluted to a final concentration of 2 mM. After 18 hours of stimulation, cells were washed and stained with anti-CD3 (OKT3, Biolegend), anti-CD69 (FN50, Biolegend), and anti-TCRα/β (IP26, Biolegend) antibodies. Cells were acquired using FACS Fortessa (BD Biosciences) automated high throughput sampler, and data analyzed using FlowJo software (Treestar).

### Example 15

### Whole-Exome Sequencing

Whole-exome sequencing of tumor DNA and matched germline leukocyte DNA was performed by inputting 75 ng of sheared genomic DNA for library preparation with the KAPA HyperPrep Kit (Roche) with modifications to the manufacturer's instruction, as described previously [57]. Library-prepared samples were captured with the SeqCap EZ MedExome Kit (NimbleGen) according to the manufacturer's instructions. Sequencing data were demultiplexed and mapped to hg19 using a custom bioinformatics pipeline, as described previously [58]. VarScan 2 [59], Mutect [60], and Strelka were used to call variants use default parameters. Variants called by at least two of the approaches were then filtered by requiring: 1) variant allele frequency of at least 2.5%, 2) at least 30× depth in both tumor and germline samples, 3) zero germline reads, and 4) a population allele frequency of less than 0.1% in the Genome Aggregation database [62].

### Example 16

### Statistical Analysis

Unless stated otherwise, all statistical analyses performed in finding high-confidence specificity groups with GLIPH2 were Fisher's exact tests using the contingency tables with the CDR3β query set (specificity group) and the reference set [24]. Poisson test was used to determine the representation bias in comparisons of distinct CDR3β sequences or specificity groups between tumors and uninvolved lungs. Student's t test was used to assess the results from all in vitro assays. Statistical significance was defined as p value <0.05.

### Example 17

Establishing Specificity Groups from Tumor-Infiltrating T Cells in Human Lung Cancer

This Example describes the results of experiments performed to identify T cells recognizing shared tumor antigens in lung cancer.

In order to identify T cells recognizing shared tumor antigens in lung cancer, specificity groups using GLIPH2 were established. As described previously, TCR clonotypes with a high probability of sharing specificities are grouped based on short amino acid sequence motifs embedded within the variable CDR3β regions of the TCR [23]. The improved GLIPH2 offers the advantage of analyzing large T cell repertoire datasets and identifying specificity groups carrying local or global sequence motifs with a much greater capacity [24]. GLIPH2 algorithms were applied to a recently published T cell repertoire dataset of 778,938 distinct CDR3β sequences from 178 HLA-typed, non-small cell lung cancer (NSCLC) patients with surgically resectable disease (see, e.g., Table 1 below). Of note, the T cell clonotypes from bulk sequencing were derived from both the surgically removed tumor as well as the uninvolved lung (see, e.g., Table 1).

TABLE 1
Summary of data available for the 178 HLA-typed
NSCLC patients from the MD Anderson Cancer Center

| Available data for the MD | | | Statistics for the MD | |
|---|---|---|---|---|
| Anderson NSCLC cohort | | | Anderson NSCLC cohort | |
| NSCLC | | Not | | NSCLC |
| (n = 178) | Available | available | Category | (n = 178) |
| Clinical data | 178 | 0 | Tumor type | 178 |
| HLA-A alleles | 177 | 1 | Adenocar- | 102 |
| HLA-B alleles | 178 | 0 | cinoma | |
| HLA-C alleles | 178 | 0 | Squamous cell | 75 |
| HLA-DRB1 alleles | 178 | 0 | carcinoma | |
| HLA-DQA1 alleles | 178 | 0 | Mixed | 1 |
| HLA-DQB1 alleles | 178 | 0 | (Adeno/SCC) | |
| Tumor-derived | 178 | 0 | Stage | |
| CDR3β | | | Stage I | 85 |
| Lung-derived | 178 | 0 | Stage II | 61 |
| CDR3β | | | Stage III | 32 |
| Exome-seq data | 91 | 87 | | |

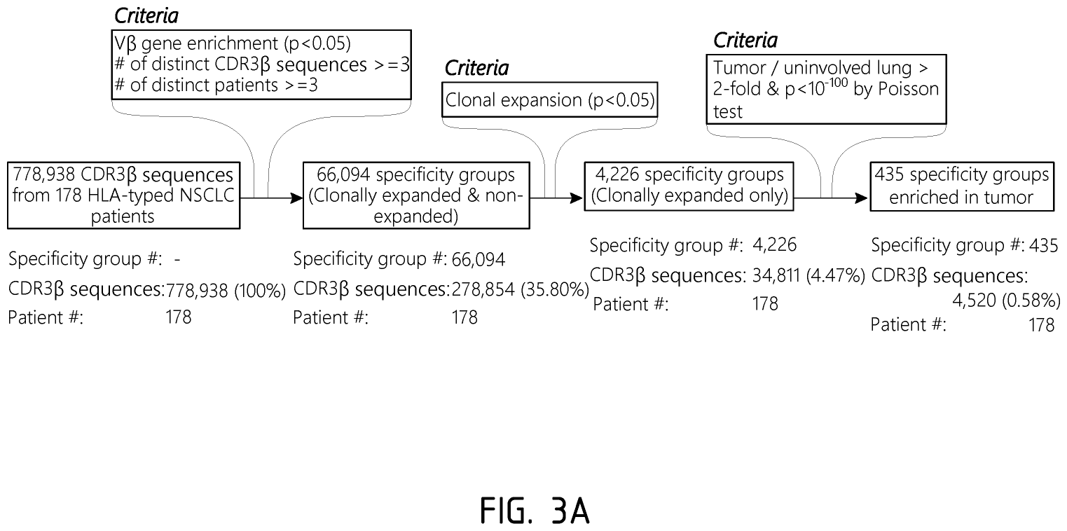

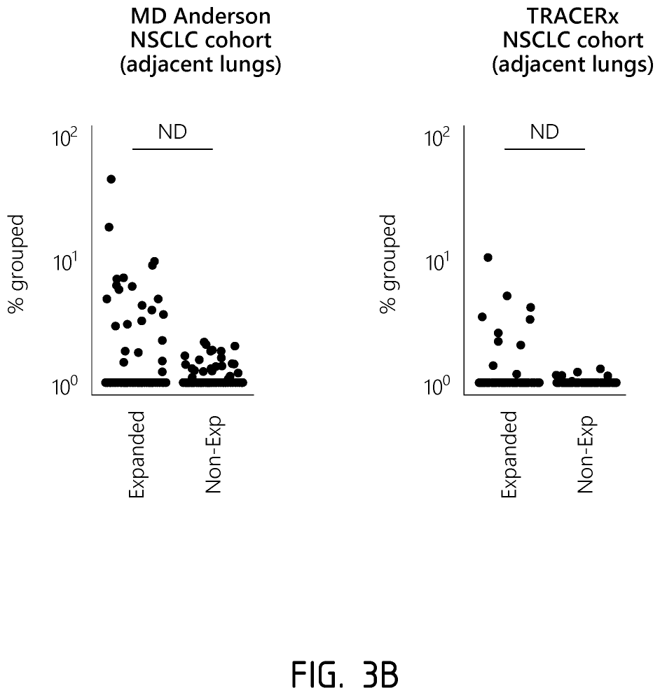

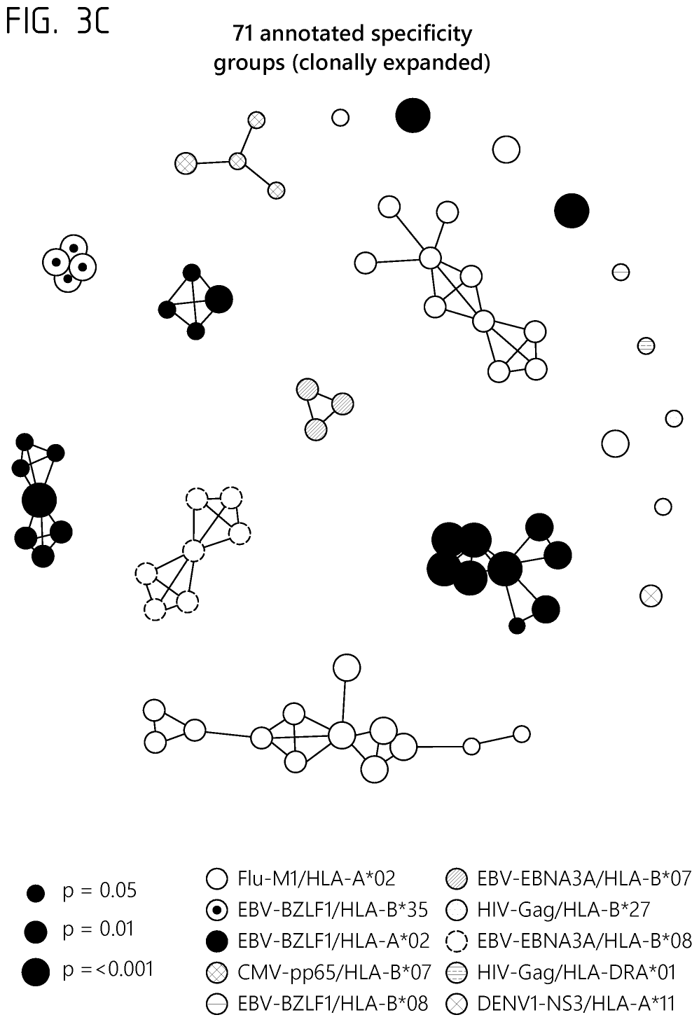

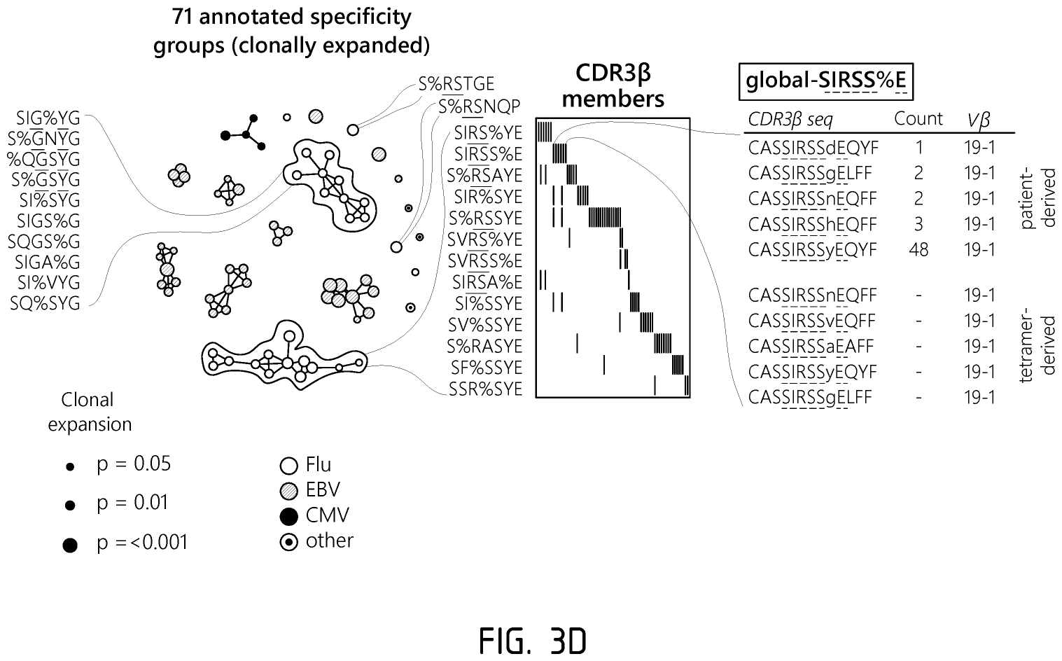

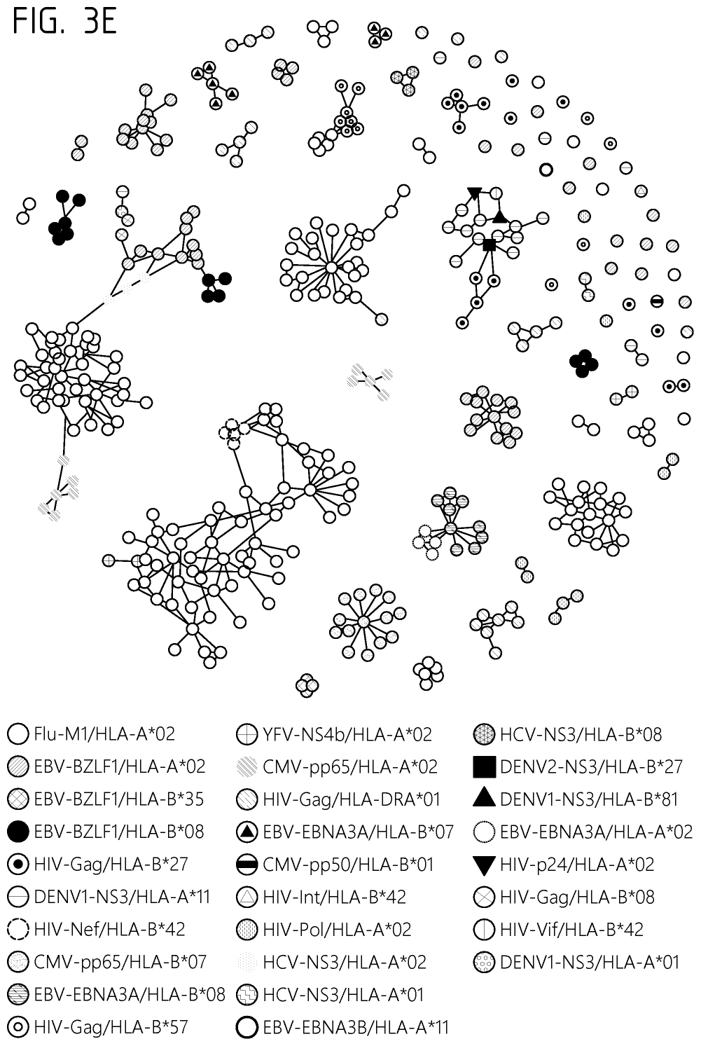

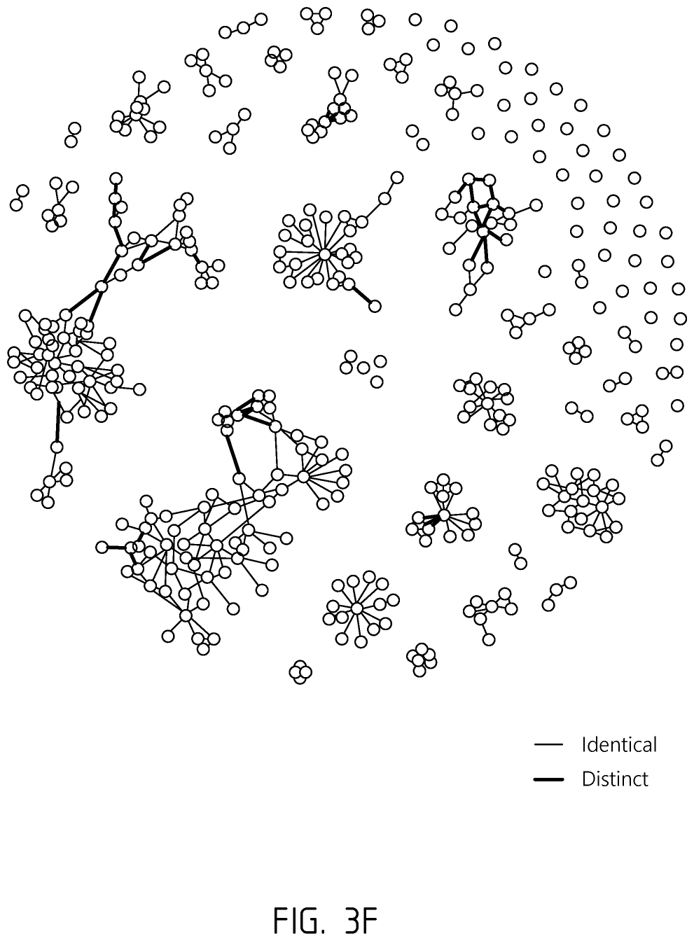

With this dataset, 435 specificity groups enriched in tumors from NSCLC patients were established after applying a set of criteria including Vβ gene enrichment and clonal expansion, the latter indicative of T cell antigen recognition (FIGS. 1A and 3A). To identify specificity groups related to antigens shared across these patients, a specificity group was further configured as including at least 3 distinct CDR3β sequences from a minimum of 3 patients. The fraction of clonotypes, which are members of the specificity groups as previously defined above, was established. It was found that significantly higher percentages of the most expanded TCR clonotypes in tumor belonged to the tumor-enriched specificity groups (FIG. 1B). In contrast, TCR clonotypes from patients' uninvolved lungs showed much lower percentages belonging to the tumor-enriched specificity groups (see, e.g., FIG. 3B). It was next established that the 435 tumor-enriched specificity groups are relevant to lung cancer, and not merely to normal lung tissue or other types of lung disease. In a validation cohort of 7,363,492 clonotypes from 202 tumor samples representing 68 NSCLC patients [26], a significantly higher percentage of top expanded TCR clonotypes in tumor belonged to the 435 tumor-enriched specificity groups compared to the non-expanded counterparts (FIG. 1B). In contrast, a lower percentage of TCR clonotypes belonging to the tumor-enriched specificity groups in lung tissue from healthy donors and patients with COPD (without a cancer diagnosis) was observed, regardless of clonal expansion (FIG. 1B). In summary, the experimental data described herein has identified a set of specificity groups predicted to recognize shared tumor antigens across NSCLC patients.

### Example 18

### In Silico Validation of TCR Specificity Groups Using HLA Tetramer Sequences

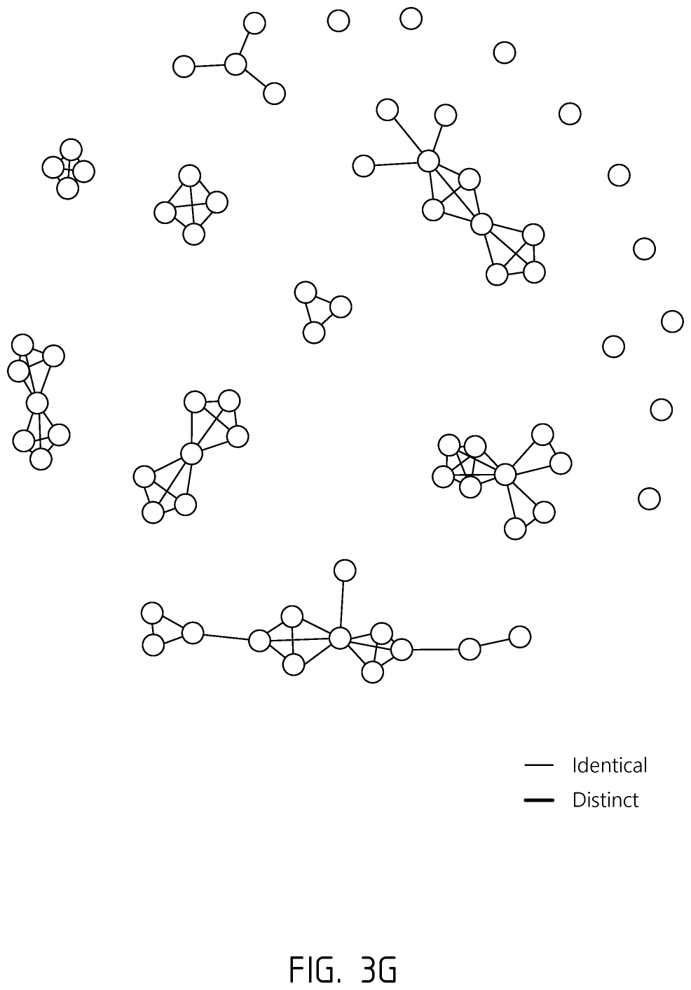

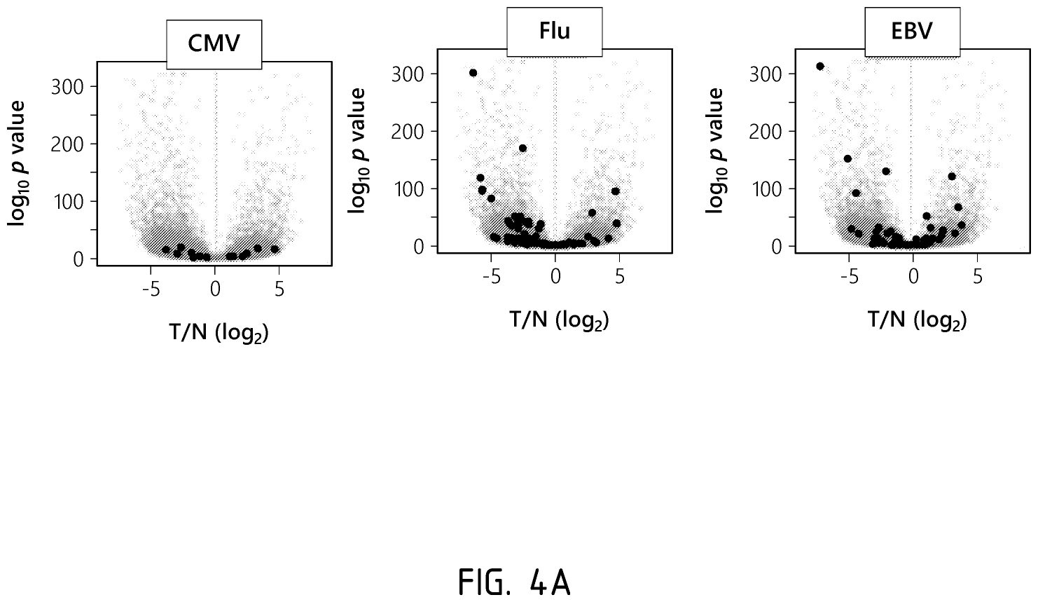

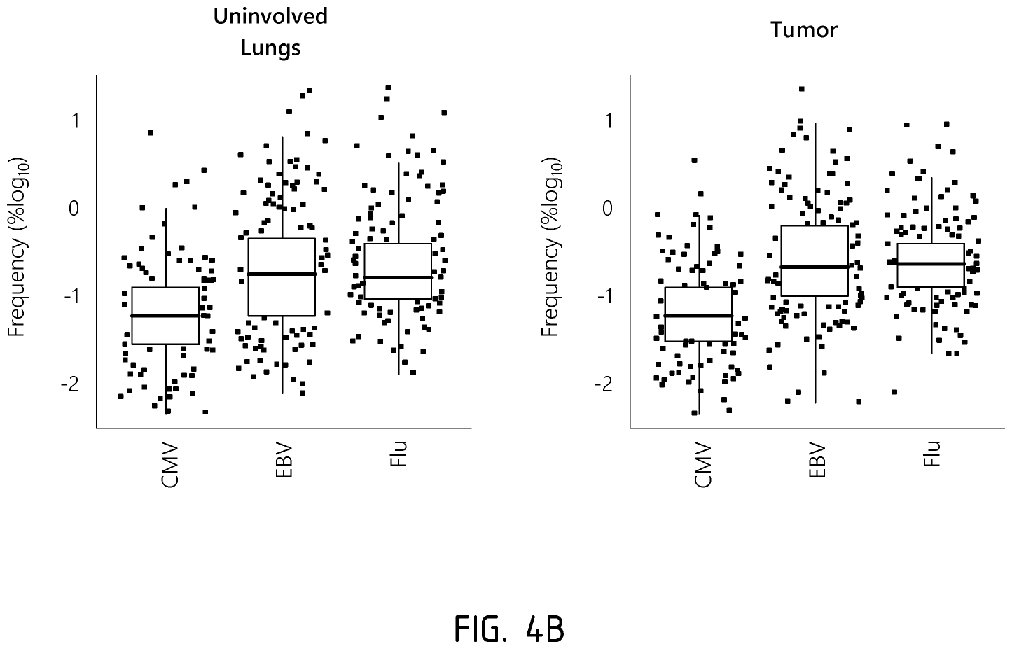

In order to validate the specificity groups established by GLIPH2, publicly available CDR3β sequences from various HLA tetramer databases were included in combination with the MD Anderson CDR3β sequences for a joint GLIPH2 analysis [23, 27, 28]. The CDR3β sequences available from the tetramer datasets primarily cover viral specificities and have been experimentally shown to bind epitopes in the context of their respective HLAs. This allows us to annotate some of the specificity groups with sequences from the tetramer databases linked to experimentally-established antigen specificities and HLA restrictions. The joint analysis led to the annotation of 396 specificity groups (FIG. 1C). Of these specificity groups, 71 were clonally expanded and annotated with 10 different tetramers (FIG. 3C). As anticipated, it was found that clonotypes with inferred specificities to Flu, Epstein-Barr virus (EBV), or CMV antigens were not preferentially localized in the tumor compared to uninvolved lung (FIGS. 4A-4B). In addition, network analysis organized these tetramer-annotated specificity groups sharing some identical CDR3β sequence members into communities (FIGS. 1C and 3D-3G). Specificity groups belonging to a given community were consistently annotated with identical HLA tetramers (FIGS. 1C, 3D, and 3E), indicating that some antigen specificity groups, albeit sharing distinct sequence motifs, are likely related to the same specificity and HLA restriction.

### Example 19

### Experimental Validation of GLIPH2-Inferred Specificities

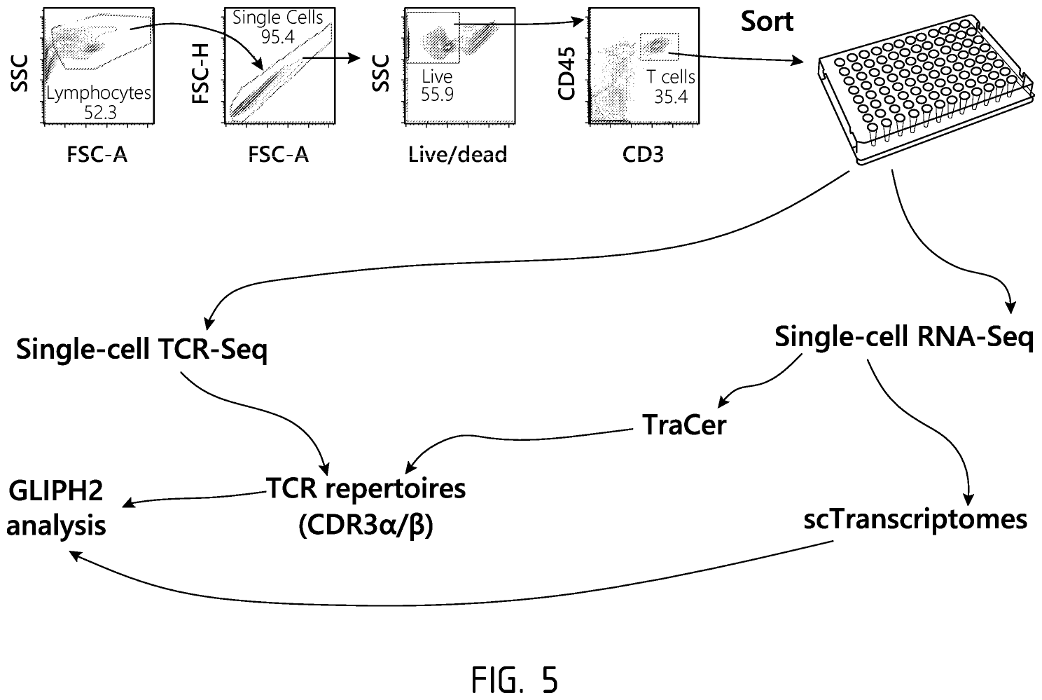

In order to experimentally validate peptide-MHC specificities, sequences from TCRα/β pairs are required from single T cells. Therefore, single-cell TCR sequencing (TCR-seq) from 15 early-stage NSCLC patients treated at Stanford was performed. Tumor-infiltrating T cells were prepared from surgically resected specimens and sorted by FACS before sequencing (see, e.g., FIG. 5). A total of 4,704 paired CDR3α and CDR3β sequences were sequenced and combined with the CDR3β sequences from MD Anderson for further analysis.

### Example 20

### Expansion of EBV-Specific T Cell Clones in Patients Responding to Immune Checkpoint Blockade

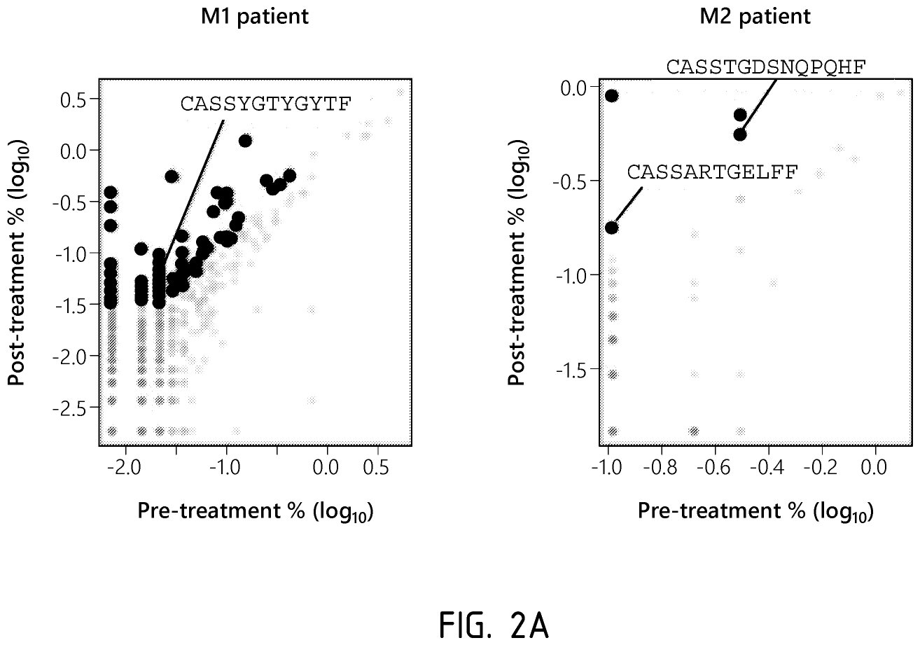

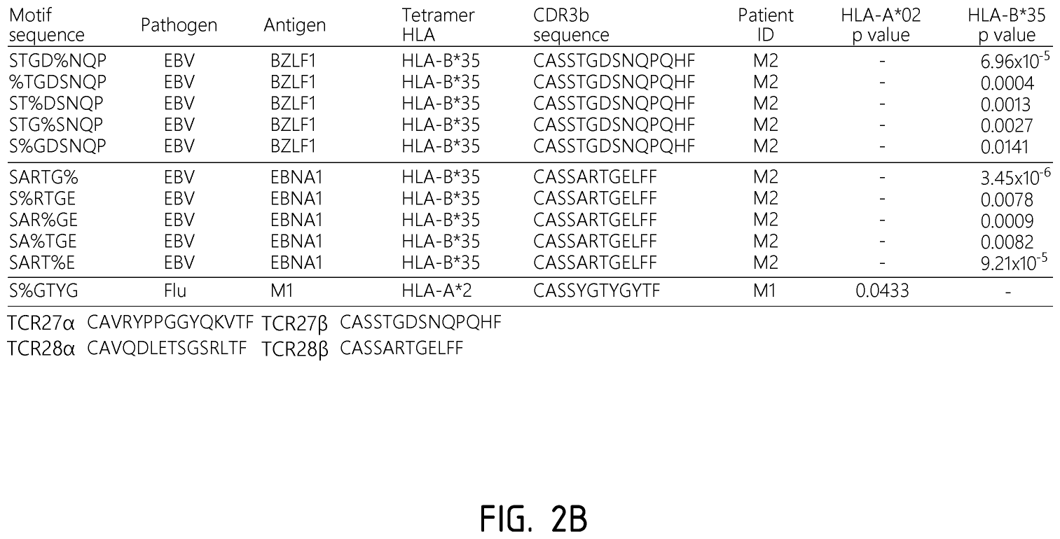

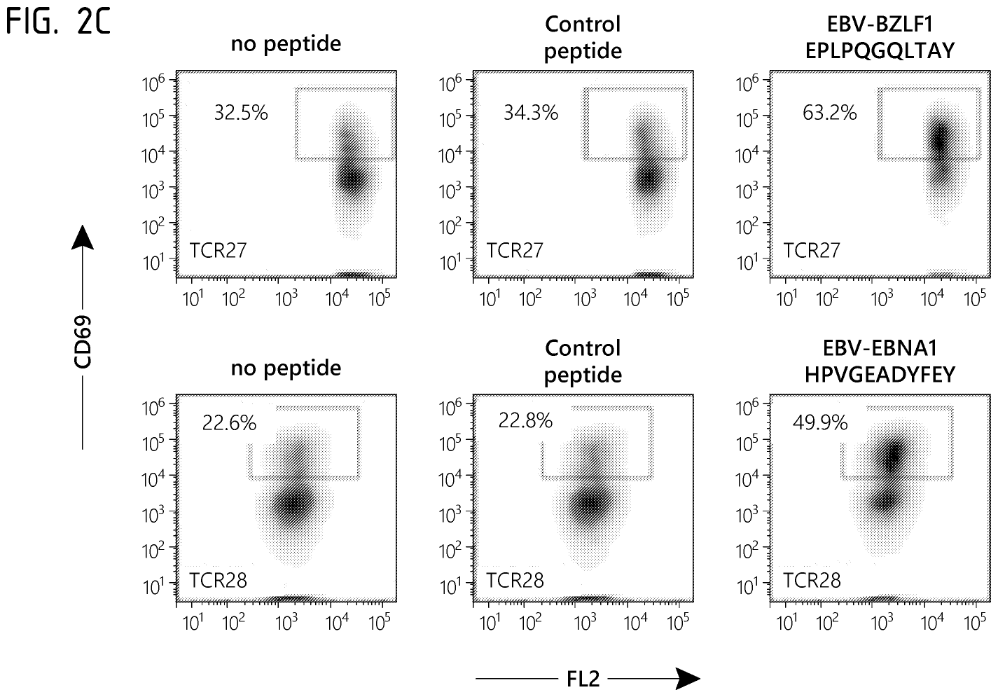

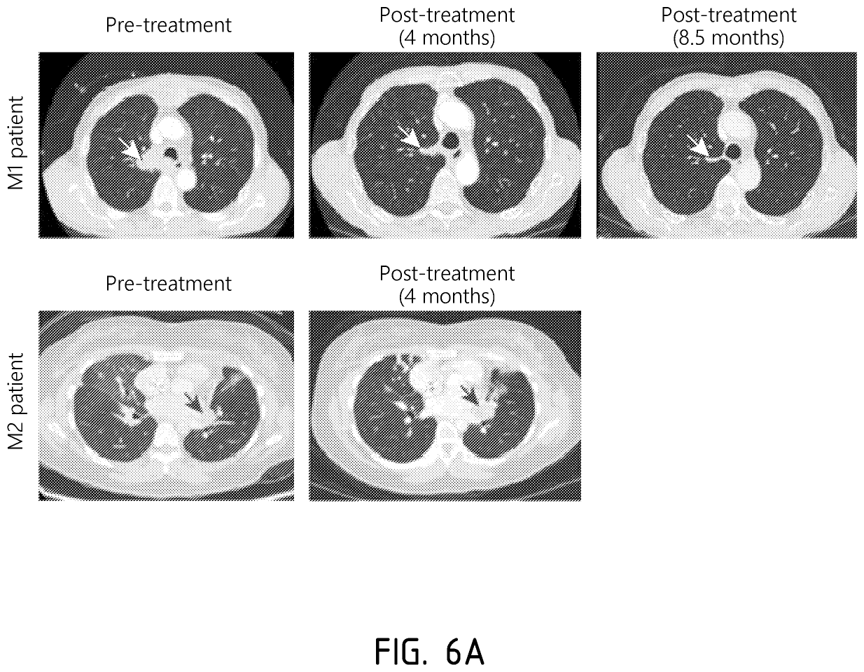

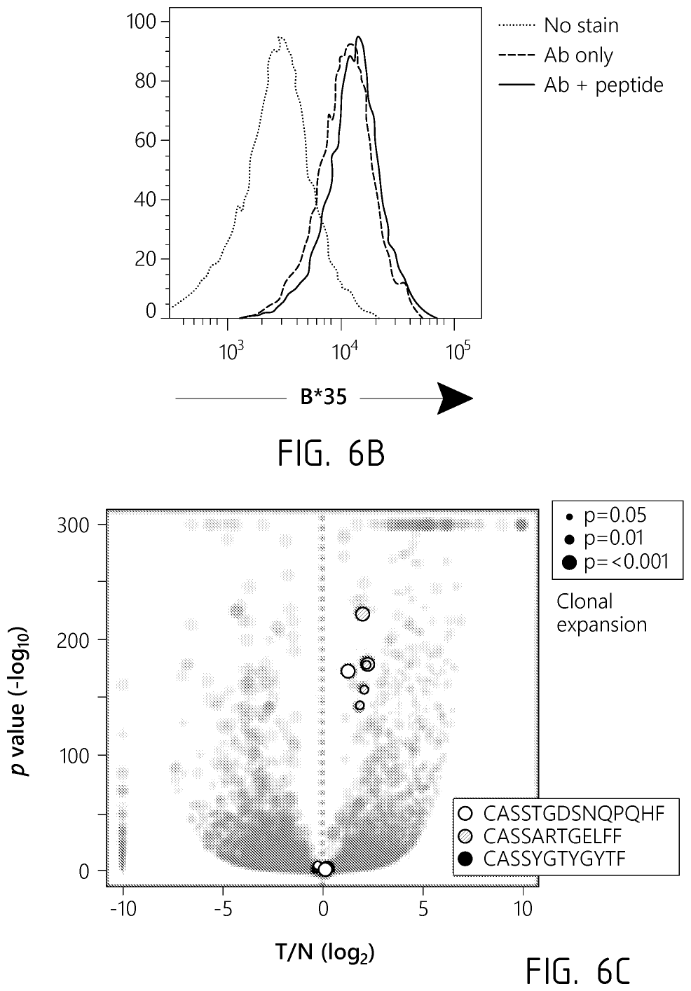

To determine if pathogen-specific T cells can impact clinical responses to anti-PD1 checkpoint immunotherapy, the TCR repertoire of two NSCLC patients who experienced a clinical response to treatment (see, e.g., FIG. 6A) was analyzed. Paired CDR3α/β repertoires on both pre- and post-treatment blood samples were sequenced and used to identify 102 CDR3β clonotypes that expanded in post-treatment samples (see, e.g., FIG. 2A). Of these expanded clones, 41 belonged to 99 specificity groups identified in tumor-infiltrating T cell CDR3β repertoires (total n=66,094, see, e.g., FIG. 3A). Tetramer CDR3β sequences were then used to annotate these specificity groups and found 11 (total n=99) containing 3 expanded CDR3β clones inferred to recognize EBV and Flu antigens (FIG. 2B). To validate the specificity inferences, two Jurkat cell clones expressing the TCRα/β chains inferred to recognize the EBV antigens and a T2 cell line expressing wildtype B*35 (FIGS. 2B and 6B) were created. Indeed, it was observed that upon co-culture with the T2-B*35 cells, both Jurkat-TCR27 and -TCR28 cells responded to the predicted EBV peptides (see, e.g., FIG. 2C). Of note, these EBV-specific specificity groups were not only expanded post-treatment, but also showed a bias in tumor compared to the adjacent lung, suggesting the potential cross-reactivities to unknown TAAs (see, e.g., FIG. 6C). Previously, common pathogen-specific T cells found in tumors have been presumed to be “bystanders” and not specific for TAAs. The experimental data described herein showed that T cell specificities for TAAs and pathogen-derived antigens were not mutually exclusive. Furthermore, these pathogen-specific T cells in tumors exhibited an effector phenotype rather than an exhausted or stressed state and lacked CD39 expression. These data suggested that cross-reactive T cells might play a role in controlling cancer progression in the setting of anti-PD1 checkpoint blockade.

While particular alternatives of the present disclosure have been disclosed, it is to be understood that various modifications and combinations are possible and are contemplated within the true spirit and scope of the appended claims. There is no intention, therefore, of limitations to the exact abstract and disclosure herein presented.

INFORMAL SEQUENCE LISTING

| SEQ | | |
|---|---|---|
| ID NO | Sequence | Description |
| 1 | CASSIRTGELFF | CDR3β TCR28 |
| 2 | CAISVRTGELFF | CDR3β TCR28 |
| 3 | CASSYRTGEAFF | CDR3β TCR28 |
| 4 | CASSYRTGELFF | CDR3β TCR28 |
| 5 | CASSLRTGEAFF | CDR3β TCR28 |
| 6 | CASSLRTGELFF | CDR3β TCR28 |
| 7 | CAWSVRTGELFF | CDR3β TCR28 |
| 8 | CASSPRTGEAFF | CDR3β TCR28 |
| 9 | CASSPRTGELFF | CDR3β TCR28 |
| 10 | CASSPRTGEQYF | CDR3β TCR28 |
| 11 | CASSRRTGEAFF | CDR3β TCR28 |
| 12 | CASSRRTGELFF | CDR3β TCR28 |
| 13 | CASSHRTGELFF | CDR3β TCR28 |
| 14 | CASSVRTGELFF | CDR3β TCR28 |
| 15 | CASSERTGEQFF | CDR3β TCR28 |
| 16 | CAWSSRTGEHTF | CDR3β TCR28 |
| 17 | CASSARTGEAFF | CDR3β TCR28 |
| 18 | CASSARTGELFF | CDR3β TCR28 |
| 19 | CASSSRTGEAFF | CDR3β TCR28 |
| 20 | CASSTRTGELFF | CDR3β TCR28 |
| 21 | CASSSRTGELFF | CDR3β TCR28 |
| 22 | CASSTRTGEAFF | CDR3β TCR28 |
| 23 | CASSGRTGELFF | CDR3β TCR28 |
| 24 | CATSGRTGELFF | CDR3β TCR28 |
| 25 | CASSQRTGEQYF | CDR3β TCR28 |
| 26 | CASSSRTGEQYF | CDR3β TCR28 |
| 27 | CASSDRTGELFF | CDR3β TCR28 |
| 28 | CASSHRTGEAFF | CDR3β TCR28 |
| 29 | CASSDRTGEAFF | CDR3β TCR28 |
| 30 | CASSASTGELFF | CDR3β TCR28 |
| 31 | CASSAGTGEAFF | CDR3β TCR28 |
| 32 | CASSAGTGELFF | CDR3β TCR28 |
| 33 | CASSAGTGEVFF | CDR3β TCR28 |
| 34 | CASSAATGELFF | CDR3β TCR28 |
| 35 | CAGSAITGELFF | CDR3β TCR28 |
| 36 | CASSANTGELFF | CDR3β TCR28 |
| 37 | CAWSAHTGELFF | CDR3β TCR28 |
| 38 | CASSAITGELFF | CDR3β TCR28 |
| 39 | CAISANTGELFF | CDR3β TCR28 |
| 40 | CASSAQTGELFF | CDR3β TCR28 |
| 41 | CAWSANTGELFF | CDR3β TCR28 |
| 42 | CASSAWTGEAFF | CDR3β TCR28 |
| 43 | CAWSAYTGELFF | CDR3β TCR28 |
| 44 | CASSAHTGELFF | CDR3β TCR28 |
| 45 | CASSAPTGELFF | CDR3β TCR28 |
| 46 | CASSAKTGELFF | CDR3β TCR28 |
| 47 | CAWSARSGELFF | CDR3β TCR28 |
| 48 | CASSARGGEQYF | CDR3β TCR28 |
| 49 | CASSARDGEAFF | CDR3β TCR28 |
| 50 | CASSARSGELFF | CDR3β TCR28 |
| 51 | CAWSARLGEOFF | CDR3β TCR28 |
| 52 | CASSARTAEQYF | CDR3β TCR28 |
| 53 | CASSARTAEAFF | CDR3β TCR28 |
| 54 | CAWSARTDEAFF | CDR3β TCR28 |
| 55 | CASSARTGTIYF | CDR3β TCR28 |
| 56 | CASSARTGDAFF | CDR3β TCR28 |
| 57 | CAIGTGDSNQPQHF | CDR3β TCR27 |
| 58 | CASGTGDSNQPQHF | CDR3β TCR27 |
| 59 | CATGTGDSNQPQHF | CDR3β TCR27 |
| 60 | CAAGTGDSNQPQHF | CDR3β TCR27 |
| 61 | CAGSTGDSNQPQHF | CDR3β TCR27 |
| 62 | CAVGTGDSNQPQHF | CDR3β TCR27 |
| 63 | CAISTGDSNQPQHF | CDR3β TCR27 |
| 64 | CASSTGDSNQPQHF | CDR3β TCR27 |
| 65 | CATSTGDSNQPQHF | CDR3β TCR27 |
| 66 | CAASTGDSNQPQHF | CDR3β TCR27 |
| 67 | CAGGTGDSNQPQHF | CDR3β TCR27 |
| 68 | CATWTGDSNQPQHF | CDR3β TCR27 |
| 69 | CSVLTGDSNQPQHF | CDR3β TCR27 |
| 70 | CAHGTGDSNQPQHF | CDR3β TCR27 |
| 71 | CASSQGDSNQPQHF | CDR3β TCR27 |
| 72 | CASSLGDSNQPQHF | CDR3β TCR27 |
| 73 | CASSIGDSNQPQHF | CDR3β TCR27 |
| 74 | CASSPGDSNQPQHF | CDR3β TCR27 |
| 75 | CXSSGGDSNQPQHF | CDR3β TCR27 |
| 76 | CASSFGDSNQPQHF | CDR3β TCR27 |
| 77 | CASSWGDSNQPQHF | CDR3β TCR27 |
| 78 | CASSAGDSNQPQHF | CDR3β TCR27 |
| 79 | CASSYGDSNQPQHF | CDR3β TCR27 |
| 80 | CASSVGDSNQPQHF | CDR3β TCR27 |
| 81 | CASSEGDSNQPQHF | CDR3β TCR27 |
| 82 | CASSRGDSNQPQHF | CDR3β TCR27 |
| 83 | CAWSSGDSNQPQHF | CDR3β TCR27 |
| 84 | CSASSGDSNQPQHF | CDR3β TCR27 |
| 85 | CASSTRDSNQPQHF | CDR3β TCR27 |
| 86 | CASSTTDSNQPQHF | CDR3β TCR27 |
| 87 | CASSTADSNQPQHF | CDR3β TCR27 |
| 88 | CASSTWDSNQPQHF | CDR3β TCR27 |
| 89 | CAWSTGSSNQPQHF | CDR3β TCR27 |
| 90 | CAWSTGGSNQPQHF | CDR3β TCR27 |
| 91 | CASSTGGSNQPQHF | CDR3β TCR27 |
| 92 | CASSTGHSNQPQHF | CDR3β TCR27 |
| 93 | CASSTGYSNQPQHF | CDR3β TCR27 |
| 94 | CASSTGNSNQPQHF | CDR3β TCR27 |
| 95 | CATSTGNSNQPQHF | CDR3β TCR27 |
| 96 | CAWSTGNSNQPQHF | CDR3β TCR27 |
| 97 | CASSTGDFNQPQHF | CDR3β TCR27 |
| 98 | CASSTGDVNQPQHF | CDR3β TCR27 |
| 99 | CAISTGDLNQPQHF | CDR3β TCR27 |
| 100 | CAISTGDGNQPQHF | CDR3β TCR27 |
| 101 | CATSTGDGNQPQHF | CDR3β TCR27 |
| 102 | CASSTGDRNQPQHF | CDR3β TCR27 |
| 103 | CATSTGDPNQPQHF | CDR3β TCR27 |
| 104 | CAISTGDANQPQHF | CDR3β TCR27 |
| 105 | CAISTGDNNQPQHF | CDR3β TCR27 |
| 106 | CASSTGDNNQPQHF | CDR3β TCR27 |
| 107 | LPFDFTPGY | control peptide |

### REFERENCES

ADDIN E N.REFLIST ADDIN E N.REFLIST 1. Kawakami, Y., et al., Cloning of the gene coding for a shared human melanoma antigen recognized by autologous T cells infiltrating into tumor. Proc Natl Acad Sci USA, 1994. 91 (9): p. 3515-9.
2. Coulie, P. G., et al., A new gene coding for a differentiation antigen recognized by autologous cytolytic T lymphocytes on HLA-A2 melanomas. J Exp Med, 1994. 180 (1): p. 35-42.
3. van der Bruggen, P., et al., A gene encoding an antigen recognized by cytolytic T lymphocytes on a human melanoma. Science, 1991. 254 (5038): p. 1643-7.
4. Coulie, P. G., et al., A mutated intron sequence codes for an antigenic peptide recognized by cytolytic T lymphocytes on a human melanoma. Proc Natl Acad Sci USA, 1995. 92 (17): p. 7976-80.
5. Wolfel, T., et al., A p16INK4a-insensitive CDK4 mutant targeted by cytolytic T lymphocytes in a human melanoma. Science, 1995. 269 (5228): p. 1281-4.
6. Murray, R. J., et al., Identification of target antigens for the human cytotoxic T cell response to Epstein-Barr virus (EBV): implications for the immune control of EBV-positive malignancies. J Exp Med, 1992. 176 (1): p. 157-68.
7. Koziel, M. J., et al., HLA class I-restricted cytotoxic T lymphocytes specific for hepatitis C virus. Identification of multiple epitopes and characterization of patterns of cytokine release. J Clin Invest, 1995. 96 (5): p. 2311-21.
8. Rehermann, B., et al., The cytotoxic T lymphocyte response to multiple hepatitis B virus polymerase epitopes during and after acute viral hepatitis. J Exp Med, 1995. 181 (3): p. 1047-58.
9. Tran, E., et al., Immunogenicity of somatic mutations in human gastrointestinal cancers. Science, 2015. 350 (6266): p. 1387-90.
10. Gros, A., et al., Prospective identification of neoantigen-specific lymphocytes in the peripheral blood of melanoma patients. Nat Med, 2016. 22 (4): p. 433-8.
11. Zacharakis, N., et al., Immune recognition of somatic mutations leading to complete durable regression in metastatic breast cancer. Nat Med, 2018. 24 (6): p. 724-730.
12. Schumacher, T. N., W. Scheper, and P. Kvistborg, Cancer Neoantigens. Annu Rev Immunol, 2019. 37: p. 173-200.
13. Rosenberg, S. A. and M. E. Dudley, Adoptive cell therapy for the treatment of patients with metastatic melanoma. Curr Opin Immunol, 2009. 21 (2): p. 233-40.
14. Hinrichs, C. S. and S. A. Rosenberg, Exploiting the curative potential of adoptive T-cell therapy for cancer. Immunol Rev, 2014. 257 (1): p. 56-71.
15. de Vos van Steenwijk, P. J., et al., An unexpectedly large polyclonal repertoire of HPV-specific T cells is poised for action in patients with cervical cancer. Cancer Res, 2010. 70 (7): p. 2707-17.
16. Piersma, S. J., et al., Human papilloma virus specific T cells infiltrating cervical cancer and draining lymph nodes show remarkably frequent use of HLA-D Q and -D P as a restriction element. Int J Cancer, 2008. 122 (3): p. 486-94.
17. Evans, E. M., et al., Infiltration of cervical cancer tissue with human papillomavirus-specific cytotoxic T-lymphocytes. Cancer Res, 1997. 57 (14): p. 2943-50.
18. Triozzi, P. L. and A. P. Fernandez, The role of the immune response in merkel cell carcinoma. Cancers (Basel), 2013. 5 (1): p. 234-54.
19. Simoni, Y., et al., Bystander CD8 (+) T cells are abundant and phenotypically distinct in human tumour infiltrates. Nature, 2018. 557 (7706): p. 575-579.
20. Rosato, P. C., et al., Virus-specific memory T cells populate tumors and can be repurposed for tumor immunotherapy. Nat Commun, 2019. 10 (1): p. 567.
21. Scheper, W., et al., Low and variable tumor reactivity of the intratumoral TCR repertoire in human cancers. Nat Med, 2019. 25 (1): p. 89-94.
22. Yu, W., et al., Clonal Deletion Prunes but Does Not Eliminate Self-Specific alphabeta CD8 (+) T Lymphocytes. Immunity, 2015. 42 (5): p. 929-41.
23. Glanville, J., et al., Identifying specificity groups in the T cell receptor repertoire. Nature, 2017. 547 (7661): p. 94-98.
24. Huang, H., et al., Analyzing the CD4+ T cell response repertoire to M. tuberculosis using GLIPH2 and whole-genome antigen screening. Nat Biotechnol, 2020.
25. Reuben, A., et al., Comprehensive T cell repertoire characterization of non-small cell lung cancer. Nat Commun, 2020. 11 (1): p. 603.
26. Joshi, K., et al., Spatial heterogeneity of the T cell receptor repertoire reflects the mutational landscape in lung cancer. Nat Med, 2019. 25 (10): p. 1549-1559.
27. Shugay, M., et al., VDJdb: a curated database of T-cell receptor sequences with known antigen specificity. Nucleic Acids Res, 2018. 46 (D1): p. D419-D427.
28. Song, I., et al., Broad TCR repertoire and diverse structural solutions for recognition of an immunodominant CD8 (+) T cell epitope. Nat Struct Mol Biol, 2017. 24 (4): p. 395-406.
29. Sidney, J., et al., HLA class I supertypes: a revised and updated classification. BMC Immunol, 2008. 9: p. 1.
30. Harjanto, S., L. F. Ng, and J. C. Tong, Clustering HLA class I superfamilies using structural interaction patterns. PLOS One, 2014. 9 (1): p.e86655.
31. Robins, H. S., et al., Overlap and effective size of the human CD8+ T cell receptor repertoire. Sci Transl Med, 2010. 2 (47): p. 47ra64.
32. Arstila, T. P., et al., A direct estimate of the human alphabeta T cell receptor diversity. Science, 1999. 286 (5441): p. 958-61.
33. Gee, M. H., et al., Antigen Identification for Orphan T Cell Receptors Expressed on Tumor-Infiltrating Lymphocytes. Cell, 2018. 172 (3): p. 549-563 e16.
34. Kheir, F., et al., Detection of Epstein-Barr Virus Infection in Non-Small Cell Lung Cancer. Cancers (Basel), 2019. 11 (6).
35. Han, A., et al., Linking T-cell receptor sequence to functional phenotype at the single-cell level. Nat Biotechnol, 2014. 32 (7): p. 684-92.
36. Stubbington, M. J. T., et al., T cell fate and clonality inference from single-cell transcriptomes. Nat Methods, 2016. 13 (4): p. 329-332.
37. Guo, X., et al., Global characterization of T cells in non-small-cell lung cancer by single-cell sequencing. Nat Med, 2018. 24 (7): p. 978-985.
38. Joglekar, A. V., et al., T cell antigen discovery via signaling and antigen-presenting bifunctional receptors. Nat Methods, 2019. 16 (2): p. 191-198.
39. Li, G., et al., T cell antigen discovery via trogocytosis. Nat Methods, 2019. 16 (2): p. 183-190.
40. Kula, T., et al., T-Scan: A Genome-wide Method for the Systematic Discovery of T Cell Epitopes. Cell, 2019. 178 (4): p. 1016-1028 e13.
41. Sewell, A. K., Why must T cells be cross-reactive? Nat Rev Immunol, 2012. 12 (9): p. 669-77.
42. McCarthy, E. F., The toxins of William B. Coley and the treatment of bone and soft-tissue sarcomas. Iowa Orthop J, 2006. 26: p. 154-8.
43. Morales, A., D. Eidinger, and A. W. Bruce, Intracavitary Bacillus Calmette-Guerin in the treatment of superficial bladder tumors. J Urol, 1976. 116 (2): p. 180-3.
44. Gopalakrishnan, V., et al., Gut microbiome modulates response to anti-PD-1 immunotherapy in melanoma patients. Science, 2018. 359 (6371): p. 97-103.
45. Routy, B., et al., Gut microbiome influences efficacy of PD-1-based immunotherapy against epithelial tumors. Science, 2018. 359 (6371): p. 91-97.
46. Vetizou, M., et al., Anticancer immunotherapy by CTLA-4 blockade relies on the gut microbiota. Science, 2015. 350 (6264): p. 1079-84.
47. Sivan, A., et al., Commensal Bifidobacterium promotes antitumor immunity and facilitates anti-P D-L1 efficacy. Science, 2015. 350 (6264): p. 1084-9.
48. Matson, V., et al., The commensal microbiome is associated with anti-PD-1 efficacy in metastatic melanoma patients. Science, 2018. 359 (6371): p. 104-108.
49. Riquelme, E., et al., Tumor Microbiome Diversity and Composition Influence Pancreatic Cancer Outcomes. Cell, 2019. 178 (4): p. 795-806 e12.
50. Emerson, R. O., et al., Immunosequencing identifies signatures of cytomegalovirus exposure history and HLA-mediated effects on the T cell repertoire. Nat Genet, 2017. 49 (5): p. 659-665.
51. Abazeed, M. E., et al., Integrative radiogenomic profiling of squamous cell lung cancer. Cancer Res, 2013. 73 (20): p. 6289-98.
52. Barbie, D. A., et al., Systematic RNA interference reveals that oncogenic KRAS-driven cancers require TBK1. Nature, 2009. 462 (7269): p. 108-12.
53. Altman, J. D. and M. M. Davis, MHC-peptide tetramers to visualize antigen-specific T cells. Curr Protoc Immunol, 2003. Chapter 17: p. Unit 17 3.
54. Picelli, S., et al., Full-length RNA-seq from single cells using Smart-seq2. Nat Protoc, 2014. 9 (1): p. 171-81.
55. Rueden, C. T., et al., ImageJ2: ImageJ for the next generation of scientific image data. BMC Bioinformatics, 2017. 18 (1): p. 529.
56. Schindelin, J., et al., Fiji: an open-source platform for biological-image analysis. Nat Methods, 2012. 9 (7): p. 676-82.
57. Hellmann, M. D., et al., Circulating tumor DNA analysis to assess risk of progression after long-term response to P D-(L) 1 blockade in NSCLC. Clin Cancer Res, 2020.
58. Newman, A. M., et al., An ultrasensitive method for quantitating circulating tumor DNA with broad patient coverage. Nat Med, 2014. 20 (5): p. 548-54.
59. Koboldt, D. C., et al., VarScan 2: somatic mutation and copy number alteration discovery in cancer by exome sequencing. Genome Res, 2012. 22 (3): p. 568-76.
60. Cibulskis, K., et al., Sensitive detection of somatic point mutations in impure and heterogeneous cancer samples. Nat Biotechnol, 2013. 31 (3): p. 213-9.
61. Saunders, C. T., et al., Strelka: accurate somatic small-variant calling from sequenced tumor-normal sample pairs. Bioinformatics, 2012. 28 (14): p. 1811-7.
62. Lek, M., et al., Analysis of protein-coding genetic variation in 60,706 humans. Nature, 2016. 536 (7616): p. 285-91.

1. An A construct comprising at least one complementary determining region (CDR) having at least 70%, 75%, 80%, 85%, 90%, 95%, 96%, 97%, 98%, 99%, or 100% sequence identity to a sequence selected from the group consisting of SEQ ID NOs: 1-106.

2. The construct of claim 1, wherein the at least one CDR has at least 70%, 75%, 80%, 85%, 90%, 95%, 96%, 97%, 98%, 99%, or 100% sequence identity to a sequence selected from the group consisting of SEQ ID NOs: 1-56.

3. The construct of claim 1, wherein the at least one CDR has at least 70%, 75%, 80%, 85%, 90%, 95%, 96%, 97%, 98%, 99%, or 100% sequence identity to a sequence selected from the group consisting of SEQ ID NOs: 57-106.

4. The construct of claim 1, wherein the construct is a single-chain construct or a double-chain construct.

5. The construct of claim 1, wherein the construct is selected from the group consisting of: (a) a T cell receptor (TCR); (b) an antibody; and (c) a functional derivative or fragment of (a) or (b).

6. The construct of claim 5, wherein the construct is a TCR construct comprising a TCR alpha chain and a TCR beta chain operably linked to each other.

7. The construct of claim 6, wherein the construct is a TCR construct comprising in its beta chain a CDR3β having at least 70%, 75%, 80%, 85%, 90%, 95%, 96%, 97%, 98%, 99%, or 100% sequence identity to a sequence selected from SEQ ID NOs: 1-106.

8. The construct of claim 7, wherein the construct further comprising in its alpha chain a CDR3α sequence.

9. The construct of claim 5, wherein construct is an antibody construct selected from the group consisting of an antigen-binding fragment (Fab), a single-chain variable fragment (scFv), a nanobody, a single domain antibody (sdAb), a V_{H} domain, a V_{L} domain, a VHH domain, a diabody, or a functional fragment of any thereof.

10. A recombinant nucleic acid comprising a nucleic acid sequence encoding a construct according to claim 1.

11. The nucleic acid of claim 10, wherein the nucleic acid sequence is operably linked to a heterologous nucleic acid sequence.

12. The nucleic acid of claim 10, wherein the nucleic acid molecule is further configured as an expression cassette or a vector.

13-14. (canceled)

15. An engineered cell comprising:
a construct according to claim 1.

16-25. (canceled)

26. A method for making an engineered cell, comprising:
a) providing a host cell capable of protein expression; and
b) transducing the provided host cell with a recombinant nucleic acid according to claim 10 to produce an engineered cell.

27. An engineered cell produced by a method according to claim 26.

28. A cell culture comprising at least one engineered cell of claim 15, and a culture medium.

29. A pharmaceutical composition comprising a pharmaceutically acceptable carrier and:
a construct according to claim 1.

30-32. (canceled)

33. A method for the prevention and/or treatment of a condition in a subject in need thereof, the method comprising administering to the subject a composition comprising:
a construct according to claim 1.

34-57. (canceled)

58. A kit for the diagnosis, prevention, and/or treatment a condition in a subject in need thereof, the system comprising:
a construct according to claim 1.

59. A method for obtaining a construct according to claim 1, the method comprising:
a) identifying a plurality of T cell receptors (TCRs) associated with a health condition;
b) determining a sequence of a CDR3β present in each of the identified TCRs; and
c) making a construct comprising a CDR3β sequence determined in (b).

60. (canceled)


SEQUENCE LISTING (154 sequences)

SEQ ID NO: 1 - protein, 12 aa, synthetic construct
  REGION 1..12: note = CDR3-Beta TCR28
CASSIRTGELFF

SEQ ID NO: 2 - protein, 12 aa, synthetic construct
  REGION 1..12: note = CDR3-Beta TCR28
CAISVRTGELFF

SEQ ID NO: 3 - protein, 12 aa, synthetic construct
  REGION 1..12: note = CDR3-Beta TCR28
CASSYRTGEAFF

SEQ ID NO: 4 - protein, 12 aa, synthetic construct
  REGION 1..12: note = CDR3-Beta TCR28
CASSYRTGELFF

SEQ ID NO: 5 - protein, 12 aa, synthetic construct
  REGION 1..12: note = CDR3-Beta TCR28
CASSLRTGEAFF

SEQ ID NO: 6 - protein, 12 aa, synthetic construct
  REGION 1..12: note = CDR3-Beta TCR28
CASSLRTGELFF

SEQ ID NO: 7 - protein, 12 aa, synthetic construct
  REGION 1..12: note = CDR3-Beta TCR28
CAWSVRTGELFF

SEQ ID NO: 8 - protein, 12 aa, synthetic construct
  REGION 1..12: note = CDR3-Beta TCR28
CASSPRTGEAFF

SEQ ID NO: 9 - protein, 12 aa, synthetic construct
  REGION 1..12: note = CDR3-Beta TCR28
CASSPRTGELFF

SEQ ID NO: 10 - protein, 12 aa, synthetic construct
  REGION 1..12: note = CDR3-Beta TCR28
CASSPRTGEQYF

SEQ ID NO: 11 - protein, 12 aa, synthetic construct
  REGION 1..12: note = CDR3-Beta TCR28
CASSRRTGEAFF

SEQ ID NO: 12 - protein, 12 aa, synthetic construct
  REGION 1..12: note = CDR3-Beta TCR28
CASSRRTGELFF

SEQ ID NO: 13 - protein, 12 aa, synthetic construct
  REGION 1..12: note = CDR3-Beta TCR28
CASSHRTGELFF

SEQ ID NO: 14 - protein, 12 aa, synthetic construct
  REGION 1..12: note = CDR3-Beta TCR28
CASSVRTGELFF

SEQ ID NO: 15 - protein, 12 aa, synthetic construct
  REGION 1..12: note = CDR3-Beta TCR28
CASSERTGEQFF

SEQ ID NO: 16 - protein, 12 aa, synthetic construct
  REGION 1..12: note = CDR3-Beta TCR28
CAWSSRTGEHTF

SEQ ID NO: 17 - protein, 12 aa, synthetic construct
  REGION 1..12: note = CDR3-Beta TCR28
CASSARTGEAFF

SEQ ID NO: 18 - protein, 12 aa, synthetic construct
  REGION 1..12: note = CDR3-Beta TCR28
CASSARTGELFF

SEQ ID NO: 19 - protein, 12 aa, synthetic construct
  REGION 1..12: note = CDR3-Beta TCR28
CASSSRTGEAFF

SEQ ID NO: 20 - protein, 12 aa, synthetic construct
  REGION 1..12: note = CDR3-Beta TCR28
CASSTRTGELFF

SEQ ID NO: 21 - protein, 12 aa, synthetic construct
  REGION 1..12: note = CDR3-Beta TCR28
CASSSRTGELFF

SEQ ID NO: 22 - protein, 12 aa, synthetic construct
  REGION 1..12: note = CDR3-Beta TCR28
CASSTRTGEAFF

SEQ ID NO: 23 - protein, 12 aa, synthetic construct
  REGION 1..12: note = CDR3-Beta TCR28
CASSGRTGELFF

SEQ ID NO: 24 - protein, 12 aa, synthetic construct
  REGION 1..12: note = CDR3-Beta TCR28
CATSGRTGELFF

SEQ ID NO: 25 - protein, 12 aa, synthetic construct
  REGION 1..12: note = CDR3-Beta TCR28
CASSQRTGEQYF

SEQ ID NO: 26 - protein, 12 aa, synthetic construct
  REGION 1..12: note = CDR3-Beta TCR28
CASSSRTGEQYF

SEQ ID NO: 27 - protein, 12 aa, synthetic construct
  REGION 1..12: note = CDR3-Beta TCR28
CASSDRTGELFF

SEQ ID NO: 28 - protein, 12 aa, synthetic construct
  REGION 1..12: note = CDR3-Beta TCR28
CASSHRTGEAFF

SEQ ID NO: 29 - protein, 12 aa, synthetic construct
  REGION 1..12: note = CDR3-Beta TCR28
CASSDRTGEAFF

SEQ ID NO: 30 - protein, 12 aa, synthetic construct
  REGION 1..12: note = CDR3-Beta TCR28
CASSASTGELFF

SEQ ID NO: 31 - protein, 12 aa, synthetic construct
  REGION 1..12: note = CDR3-Beta TCR28
CASSAGTGEAFF

SEQ ID NO: 32 - protein, 12 aa, synthetic construct
  REGION 1..12: note = CDR3-Beta TCR28
CASSAGTGELFF

SEQ ID NO: 33 - protein, 12 aa, synthetic construct
  REGION 1..12: note = CDR3-Beta TCR28
CASSAGTGEVFF

SEQ ID NO: 34 - protein, 12 aa, synthetic construct
  REGION 1..12: note = CDR3-Beta TCR28
CASSAATGELFF

SEQ ID NO: 35 - protein, 12 aa, synthetic construct
  REGION 1..12: note = CDR3-Beta TCR28
CAGSAITGELFF

SEQ ID NO: 36 - protein, 12 aa, synthetic construct
  REGION 1..12: note = CDR3-Beta TCR28
CASSANTGELFF

SEQ ID NO: 37 - protein, 12 aa, synthetic construct
  REGION 1..12: note = CDR3-Beta TCR28
CAWSAHTGELFF

SEQ ID NO: 38 - protein, 12 aa, synthetic construct
  REGION 1..12: note = CDR3-Beta TCR28
CASSAITGELFF

SEQ ID NO: 39 - protein, 12 aa, synthetic construct
  REGION 1..12: note = CDR3-Beta TCR28
CAISANTGELFF

SEQ ID NO: 40 - protein, 12 aa, synthetic construct
  REGION 1..12: note = CDR3-Beta TCR28
CASSAQTGELFF

SEQ ID NO: 41 - protein, 12 aa, synthetic construct
  REGION 1..12: note = CDR3-Beta TCR28
CAWSANTGELFF

SEQ ID NO: 42 - protein, 12 aa, synthetic construct
  REGION 1..12: note = CDR3-Beta TCR28
CASSAWTGEAFF

SEQ ID NO: 43 - protein, 12 aa, synthetic construct
  REGION 1..12: note = CDR3-Beta TCR28
CAWSAYTGELFF

SEQ ID NO: 44 - protein, 12 aa, synthetic construct
  REGION 1..12: note = CDR3-Beta TCR28
CASSAHTGELFF

SEQ ID NO: 45 - protein, 12 aa, synthetic construct
  REGION 1..12: note = CDR3-Beta TCR28
CASSAPTGELFF

SEQ ID NO: 46 - protein, 12 aa, synthetic construct
  REGION 1..12: note = CDR3-Beta TCR28
CASSAKTGELFF

SEQ ID NO: 47 - protein, 12 aa, synthetic construct
  REGION 1..12: note = CDR3-Beta TCR28
CAWSARSGELFF

SEQ ID NO: 48 - protein, 12 aa, synthetic construct
  REGION 1..12: note = CDR3-Beta TCR28
CASSARGGEQYF

SEQ ID NO: 49 - protein, 12 aa, synthetic construct
  REGION 1..12: note = CDR3-Beta TCR28
CASSARDGEAFF

SEQ ID NO: 50 - protein, 12 aa, synthetic construct
  REGION 1..12: note = CDR3-Beta TCR28
CASSARSGELFF

SEQ ID NO: 51 - protein, 12 aa, synthetic construct
  REGION 1..12: note = CDR3-Beta TCR28
CAWSARLGEQFF

SEQ ID NO: 52 - protein, 12 aa, synthetic construct
  REGION 1..12: note = CDR3-Beta TCR28
CASSARTAEQYF

SEQ ID NO: 53 - protein, 12 aa, synthetic construct
  REGION 1..12: note = CDR3-Beta TCR28
CASSARTAEAFF

SEQ ID NO: 54 - protein, 12 aa, synthetic construct
  REGION 1..12: note = CDR3-Beta TCR28
CAWSARTDEAFF

SEQ ID NO: 55 - protein, 12 aa, synthetic construct
  REGION 1..12: note = CDR3-Beta TCR28
CASSARTGTIYF

SEQ ID NO: 56 - protein, 12 aa, synthetic construct
  REGION 1..12: note = CDR3-Beta TCR28
CASSARTGDAFF

SEQ ID NO: 57 - protein, 14 aa, synthetic construct
  REGION 1..14: note = CDR3-Beta TCR27
CAIGTGDSNQPQHF

SEQ ID NO: 58 - protein, 14 aa, synthetic construct
  REGION 1..14: note = CDR3-Beta TCR27
CASGTGDSNQPQHF

SEQ ID NO: 59 - protein, 14 aa, synthetic construct
  REGION 1..14: note = CDR3-Beta TCR27
CATGTGDSNQPQHF

SEQ ID NO: 60 - protein, 14 aa, synthetic construct
  REGION 1..14: note = CDR3-Beta TCR27
CAAGTGDSNQPQHF

SEQ ID NO: 61 - protein, 14 aa, synthetic construct
  REGION 1..14: note = CDR3-Beta TCR27
CAGSTGDSNQPQHF

SEQ ID NO: 62 - protein, 14 aa, synthetic construct
  REGION 1..14: note = CDR3-Beta TCR27
CAVGTGDSNQPQHF

SEQ ID NO: 63 - protein, 14 aa, synthetic construct
  REGION 1..14: note = CDR3-Beta TCR27
CAISTGDSNQPQHF

SEQ ID NO: 64 - protein, 14 aa, synthetic construct
  REGION 1..14: note = CDR3-Beta TCR27
CASSTGDSNQPQHF

SEQ ID NO: 65 - protein, 14 aa, synthetic construct
  REGION 1..14: note = CDR3-Beta TCR27
CATSTGDSNQPQHF

SEQ ID NO: 66 - protein, 14 aa, synthetic construct
  REGION 1..14: note = CDR3-Beta TCR27
CAASTGDSNQPQHF

SEQ ID NO: 67 - protein, 14 aa, synthetic construct
  REGION 1..14: note = CDR3-Beta TCR27
CAGGTGDSNQPQHF

SEQ ID NO: 68 - protein, 14 aa, synthetic construct
  REGION 1..14: note = CDR3-Beta TCR27
CATWTGDSNQPQHF

SEQ ID NO: 69 - protein, 14 aa, synthetic construct
  REGION 1..14: note = CDR3-Beta TCR27
CSVLTGDSNQPQHF

SEQ ID NO: 70 - protein, 14 aa, synthetic construct
  REGION 1..14: note = CDR3-Beta TCR27
CAHGTGDSNQPQHF

SEQ ID NO: 71 - protein, 14 aa, synthetic construct
  REGION 1..14: note = CDR3-Beta TCR27
CASSQGDSNQPQHF

SEQ ID NO: 72 - protein, 14 aa, synthetic construct
  REGION 1..14: note = CDR3-Beta TCR27
CASSLGDSNQPQHF

SEQ ID NO: 73 - protein, 14 aa, synthetic construct
  REGION 1..14: note = CDR3-Beta TCR27
CASSIGDSNQPQHF

SEQ ID NO: 74 - protein, 14 aa, synthetic construct
  REGION 1..14: note = CDR3-Beta TCR27
CASSPGDSNQPQHF

SEQ ID NO: 75 - protein, 14 aa, synthetic construct
  REGION 1..14: note = CDR3-Beta TCR27
  VARIANT 2: note = X can be any amino acid
CXSSGGDSNQPQHF

SEQ ID NO: 76 - protein, 14 aa, synthetic construct
  REGION 1..14: note = CDR3-Beta TCR27
CASSFGDSNQPQHF

SEQ ID NO: 77 - protein, 14 aa, synthetic construct
  REGION 1..14: note = CDR3-Beta TCR27
CASSWGDSNQPQHF

SEQ ID NO: 78 - protein, 14 aa, synthetic construct
  REGION 1..14: note = CDR3-Beta TCR27
CASSAGDSNQPQHF

SEQ ID NO: 79 - protein, 14 aa, synthetic construct
  REGION 1..14: note = CDR3-Beta TCR27
CASSYGDSNQPQHF

SEQ ID NO: 80 - protein, 14 aa, synthetic construct
  REGION 1..14: note = CDR3-Beta TCR27
CASSVGDSNQPQHF

SEQ ID NO: 81 - protein, 14 aa, synthetic construct
  REGION 1..14: note = CDR3-Beta TCR27
CASSEGDSNQPQHF

SEQ ID NO: 82 - protein, 14 aa, synthetic construct
  REGION 1..14: note = CDR3-Beta TCR27
CASSRGDSNQPQHF

SEQ ID NO: 83 - protein, 14 aa, synthetic construct
  REGION 1..14: note = CDR3-Beta TCR27
CAWSSGDSNQPQHF

SEQ ID NO: 84 - protein, 14 aa, synthetic construct
  REGION 1..14: note = CDR3-Beta TCR27
CSASSGDSNQPQHF

SEQ ID NO: 85 - protein, 14 aa, synthetic construct
  REGION 1..14: note = CDR3-Beta TCR27
CASSTRDSNQPQHF

SEQ ID NO: 86 - protein, 14 aa, synthetic construct
  REGION 1..14: note = CDR3-Beta TCR27
CASSTTDSNQPQHF

SEQ ID NO: 87 - protein, 14 aa, synthetic construct
  REGION 1..14: note = CDR3-Beta TCR27
CASSTADSNQPQHF

SEQ ID NO: 88 - protein, 14 aa, synthetic construct
  REGION 1..14: note = CDR3-Beta TCR27
CASSTWDSNQPQHF

SEQ ID NO: 89 - protein, 14 aa, synthetic construct
  REGION 1..14: note = CDR3-Beta TCR27
CAWSTGSSNQPQHF

SEQ ID NO: 90 - protein, 14 aa, synthetic construct
  REGION 1..14: note = CDR3-Beta TCR27
CAWSTGGSNQPQHF

SEQ ID NO: 91 - protein, 14 aa, synthetic construct
  REGION 1..14: note = CDR3-Beta TCR27
CASSTGGSNQPQHF

SEQ ID NO: 92 - protein, 14 aa, synthetic construct
  REGION 1..14: note = CDR3-Beta TCR27
CASSTGHSNQPQHF

SEQ ID NO: 93 - protein, 14 aa, synthetic construct
  REGION 1..14: note = CDR3-Beta TCR27
CASSTGYSNQPQHF

SEQ ID NO: 94 - protein, 14 aa, synthetic construct
  REGION 1..14: note = CDR3-Beta TCR27
CASSTGNSNQPQHF

SEQ ID NO: 95 - protein, 14 aa, synthetic construct
  REGION 1..14: note = CDR3-Beta TCR27
CATSTGNSNQPQHF

SEQ ID NO: 96 - protein, 14 aa, synthetic construct
  REGION 1..14: note = CDR3-Beta TCR27
CAWSTGNSNQPQHF

SEQ ID NO: 97 - protein, 14 aa, synthetic construct
  REGION 1..14: note = CDR3-Beta TCR27
CASSTGDFNQPQHF

SEQ ID NO: 98 - protein, 14 aa, synthetic construct
  REGION 1..14: note = CDR3-Beta TCR27
CASSTGDVNQPQHF

SEQ ID NO: 99 - protein, 14 aa, synthetic construct
  REGION 1..14: note = CDR3-Beta TCR27
CAISTGDLNQPQHF

SEQ ID NO: 100 - protein, 14 aa, synthetic construct
  REGION 1..14: note = CDR3-Beta TCR27
CAISTGDGNQPQHF

SEQ ID NO: 101 - protein, 14 aa, synthetic construct
  REGION 1..14: note = CDR3-Beta TCR27
CATSTGDGNQPQHF

SEQ ID NO: 102 - protein, 14 aa, synthetic construct
  REGION 1..14: note = CDR3-Beta TCR27
CASSTGDRNQPQHF

SEQ ID NO: 103 - protein, 14 aa, synthetic construct
  REGION 1..14: note = CDR3-Beta TCR27
CATSTGDPNQPQHF

SEQ ID NO: 104 - protein, 14 aa, synthetic construct
  REGION 1..14: note = CDR3-Beta TCR27
CAISTGDANQPQHF

SEQ ID NO: 105 - protein, 14 aa, synthetic construct
  REGION 1..14: note = CDR3-Beta TCR27
CAISTGDNNQPQHF

SEQ ID NO: 106 - protein, 14 aa, synthetic construct
  REGION 1..14: note = CDR3-Beta TCR27
CASSTGDNNQPQHF

SEQ ID NO: 107 - protein, 9 aa, synthetic construct
  REGION 1..9: note = control peptide
LPFDFTPGY

SEQ ID NO: 108 - protein, 8 aa, synthetic construct
  REGION 1..8: note = CDR3 Beta TCR27 motif consensus
  VARIANT 5: note = X can be any amino acid
STGDXNQP

SEQ ID NO: 109 - protein, 8 aa, synthetic construct
  REGION 1..8: note = CDR3 Beta TCR27 motif consensus
  VARIANT 1: note = X can be any amino acid
XTGDSNQP

SEQ ID NO: 110 - protein, 8 aa, synthetic construct
  REGION 1..8: note = CDR3 Beta TCR27 motif consensus
  VARIANT 3: note = X can be any amino acid
STXDSNQP

SEQ ID NO: 111 - protein, 8 aa, synthetic construct
  REGION 1..8: note = CDR3 Beta TCR27 motif consensus
  VARIANT 4: note = X can be any amino acid
STGXSNQP

SEQ ID NO: 112 - protein, 8 aa, synthetic construct
  REGION 1..8: note = CDR3 Beta TCR27 motif consensus
  VARIANT 2: note = X can be any amino acid
SXGDSNQP

SEQ ID NO: 113 - protein, 6 aa, synthetic construct
  REGION 1..6: note = CDR3 Beta TCR28 motif consensus
  VARIANT 6: note = X can be any amino acid
SARTGX

SEQ ID NO: 114 - protein, 6 aa, synthetic construct
  REGION 1..6: note = CDR3 Beta TCR28 motif consensus
  VARIANT 2: note = X can be any amino acid
SXRTGE

SEQ ID NO: 115 - protein, 6 aa, synthetic construct
  REGION 1..6: note = CDR3 Beta TCR28 motif consensus
  VARIANT 4: note = X can be any amino acid
SARXGE

SEQ ID NO: 116 - protein, 6 aa, synthetic construct
  REGION 1..6: note = CDR3 Beta TCR28 motif consensus
  VARIANT 3: note = X can be any amino acid
SAXTGE

SEQ ID NO: 117 - protein, 6 aa, synthetic construct
  REGION 1..6: note = CDR3 Beta TCR28 motif consensus
  VARIANT 5: note = X can be any amino acid
SARTXE

SEQ ID NO: 118 - protein, 12 aa, synthetic construct
CASSYGTYGYTF

SEQ ID NO: 119 - protein, 6 aa, synthetic construct
  VARIANT 4: note = X can be any amino acid
SIGXYG

SEQ ID NO: 120 - protein, 6 aa, synthetic construct
  VARIANT 2: note = X can be any amino acid
SXGNYG

SEQ ID NO: 121 - protein, 6 aa, synthetic construct
  VARIANT 1: note = X can be any amino acid
XQGSYG

SEQ ID NO: 122 - protein, 6 aa, synthetic construct
  VARIANT 2: note = X can be any amino acid
SXGSYG

SEQ ID NO: 123 - protein, 6 aa, synthetic construct
  VARIANT 3: note = X can be any amino acid
SIXSYG

SEQ ID NO: 124 - protein, 6 aa, synthetic construct
  VARIANT 5: note = X can be any amino acid
SIGSXG

SEQ ID NO: 125 - protein, 6 aa, synthetic construct
  VARIANT 5: note = X can be any amino acid
SQGSXG

SEQ ID NO: 126 - protein, 6 aa, synthetic construct
  VARIANT 5: note = X can be any amino acid
SIGAXG

SEQ ID NO: 127 - protein, 6 aa, synthetic construct
  VARIANT 3: note = X can be any amino acid
SIXVYG

SEQ ID NO: 128 - protein, 6 aa, synthetic construct
  VARIANT 3: note = X can be any amino acid
SQXSYG

SEQ ID NO: 129 - protein, 7 aa, synthetic construct
  VARIANT 2: note = X can be any amino acid
SXRSTGE

SEQ ID NO: 130 - protein, 7 aa, synthetic construct
  VARIANT 2: note = X can be any amino acid
SXRSNQP

SEQ ID NO: 131 - protein, 7 aa, synthetic construct
  VARIANT 5: note = X can be any amino acid
SIRSXYE

SEQ ID NO: 132 - protein, 7 aa, synthetic construct
  VARIANT 6: note = X can be any amino acid
SIRSSXE

SEQ ID NO: 133 - protein, 7 aa, synthetic construct
  VARIANT 2: note = X can be any amino acid
SXRSAYE

SEQ ID NO: 134 - protein, 7 aa, synthetic construct
  VARIANT 4: note = X can be any amino acid
SIRXSYE

SEQ ID NO: 135 - protein, 7 aa, synthetic construct
  VARIANT 2: note = X can be any amino acid
SXRSSYE

SEQ ID NO: 136 - protein, 7 aa, synthetic construct
  VARIANT 5: note = X can be any amino acid
SVRSXYE

SEQ ID NO: 137 - protein, 7 aa, synthetic construct
  VARIANT 6: note = X can be any amino acid
SVRSSXE

SEQ ID NO: 138 - protein, 7 aa, synthetic construct
  VARIANT 6: note = X can be any amino acid
SIRSAXE

SEQ ID NO: 139 - protein, 7 aa, synthetic construct
  VARIANT 3: note = X can be any amino acid
SIXSSYE

SEQ ID NO: 140 - protein, 7 aa, synthetic construct
  VARIANT 3: note = X can be any amino acid
SVXSSYE

SEQ ID NO: 141 - protein, 7 aa, synthetic construct
  VARIANT 2: note = X can be any amino acid
SXRASYE

SEQ ID NO: 142 - protein, 7 aa, synthetic construct
  VARIANT 3: note = X can be any amino acid
SFXSSYE

SEQ ID NO: 143 - protein, 7 aa, synthetic construct
  VARIANT 4: note = X can be any amino acid
SSRXSYE

SEQ ID NO: 144 - protein, 13 aa, synthetic construct
CASSIRSSDEQYF

SEQ ID NO: 145 - protein, 13 aa, synthetic construct
CASSIRSSGELFF

SEQ ID NO: 146 - protein, 13 aa, synthetic construct
CASSIRSSNEQFF

SEQ ID NO: 147 - protein, 13 aa, synthetic construct
CASSIRSSHEQFF

SEQ ID NO: 148 - protein, 13 aa, synthetic construct
CASSIRSSYEQYF

SEQ ID NO: 149 - protein, 13 aa, synthetic construct
CASSIRSSVEQFF

SEQ ID NO: 150 - protein, 13 aa, synthetic construct
CASSIRSSAEAFF

SEQ ID NO: 151 - protein, 11 aa, synthetic construct
EPLPQGQLTAY

SEQ ID NO: 152 - protein, 11 aa, synthetic construct
HPVGEADYFEY

SEQ ID NO: 153 - protein, 15 aa, synthetic construct
CAVRYPPGGYQKVTF

SEQ ID NO: 154 - protein, 15 aa, synthetic construct
CAVQDLETSGSRLTF